SEQUENCE LISTING (19 sequences)

SEQ ID NO: 1 - protein, 8 aa, Artificial Sequence
  Synthetic peptide
RERRRKKR

SEQ ID NO: 2 - DNA, 24 bp, Artificial Sequence
  Synthetic primer
ggcacctataaattgggctcaagg

SEQ ID NO: 3 - DNA, 38 bp, Artificial Sequence
  Synthetic primer
cttttttcttcttctctctctagattgaatagacggga

SEQ ID NO: 4 - DNA, 22 bp, Artificial Sequence
  Synthetic primer
aatttcactaaagctgcggccg

SEQ ID NO: 5 - DNA, 34 bp, Artificial Sequence
  Synthetic primer
gagagaagaagaaaaaagagaggcctatttgggg

SEQ ID NO: 6 - DNA, 22 bp, Artificial Sequence
  Synthetic primer
gaaggccgtcaaggccgcatgg

SEQ ID NO: 7 - DNA, 45 bp, Artificial Sequence
  Synthetic primer
tctcttttttcttcttctctctcttgattcaatctggggaacatt

SEQ ID NO: 8 - DNA, 24 bp, Artificial Sequence
  Synthetic primer
gcagtgaaaggaaggcccatgagg

SEQ ID NO: 9 - DNA, 41 bp, Artificial Sequence
  Synthetic primer
gagagaagaagaaaaaagagaggattgtttggggcaatagc

SEQ ID NO: 10 - DNA, 39 bp, Influenza virus
attcaatctagagagagaagaagaaaaaagagaggccta

SEQ ID NO: 11 - protein, 13 aa, Influenza virus
IQSRERRRKKRGL

SEQ ID NO: 12 - DNA, 39 bp, Influenza virus
attgaatcaagagagagaagaagaaaaaagagaggattg

SEQ ID NO: 13 - protein, 13 aa, Influenza virus
IESRERRRKKRGL

SEQ ID NO: 14 - protein, 573 aa, Influenza virus
MKAILVVLLYTFATANADTLCIGYHANNSTDTVDTVLEKNVTVTHSVNLLEDKHNGKLCKLRGVAPLHLGKCNIAGWILGNPECESLSTASSWSYIVETPSSDNGTCYPGDFIDYEELREQLSSVSSFERFEIFPKTSSWPNHDSNKGVTAACPHAGAKSFYKNLIWLVKKGNSYPKLSKSYINDKGKEVLVLWGIHHPSTSADQQSLYQNADTYVFVGSSRYSKKFKPEIAIRPKVRDQEGRMNYYWTLVEPGDKITFEATGNLVVPRYAFAMERNAGSGIIISDTPVHDCNTTCQTPKGAINTSLPFQNIHPITIGKCPKYVKSTKLRLATGLRNIPSIQSRERRRKKRGLFGAIAGFIEGGWTGMVDGWYGYHHQNEQGSGYAADLKSTQNAIDEITNKVNSVIEKMNTQFTAVGKEFNHLEKRIENLNKKVDDGFLDIWTYNAELLVLLENERTLDYHDSNVKNLYEKVRSQLKNNAKEIGNGCFEFYHKCDNTCMESVKNGTYDYPKYSEEAKLNREEIDGVKLESTRIYQILAIYSTVASSLVLVVSLGAISFWMCSNGSLQCRICI

SEQ ID NO: 15 - protein, 569 aa, Influenza virus
MAIIYLILLFTAVRGDQICIGYHANNSTEKVDTILERNVTVTHAKDILEKTHNGKLCKLNGIPPLELGDCSIAGWLLGNPECDRLLSVPEWSYIMEKENPRDGLCYPGSFNDYEELKHLLSSVKHFEKVKILPKDRWTQHTTTGGSRACAVSGNPSFFRNMVWLTKKGSNYPVAKGSYNNTSGEQMLIIWGVHHPNDETEQRTLYQNVGTYVSVGTSTLNKRSTPDIATRPKVNGLGSRMEFSWTLLDMWDTINFESTGNLIAPEYGFKISKRGSSGIMKTEGTLENCETKCQTPLGAINTTLPFHNVHPLTIGECPKYVKSEKLVLATGLRNVPQIQSRERRRKKRGLFGAIAGFIEGGWQGMVDGWYGYHHSNDQGSGYAADKESTQKAFDGITNKVNSVIEKMNTQFEAVGKEFSNLERRLENLNKKMEDGFLDVWTYNAELLVLMENERTLDFHDSNVKNLYDKVRMQLRDNVKELGNGCFEFYHKCDDECMNSVKNGTYDYPKYEEESKLNRNEIKGVKLSSMGVYQILAIYATVAGSLSLAIMMAGISFWMCSNGSLQCRICI

SEQ ID NO: 16 - protein, 566 aa, Influenza virus
MKAILVVLLYTFATANADTLCIGYHANNSTDTVDTVLEKNVTVTHSVNLLEDKHNGKLCKLRGVAPLHLGKCNIAGWILGNPECESLSTASSWSYIVETPSSDNGTCYPGDFIDYEELREQLSSVSSFERFEIFPKTSSWPNHDSNKGVTAACPHAGAKSFYKNLIWLVKKGNSYPKLSKSYINDKGKEVLVLWGIHHPSTSADQQSLYQNADTYVFVGSSRYSKKFKPEIAIRPKVRDQEGRMNYYWTLVEPGDKITFEATGNLVVPRYAFAMERNAGSGIIISDTPVHDCNTTCQTPKGAINTSLPFQNIHPITIGKCPKYVKSTKLRLATGLRNIPSIQSRGLFGAIAGFIEGGWTGMVDGWYGYHHQNEQGSGYAADLKSTQNAIDEITNKVNSVIEKMNTQFTAVGKEFNHLEKRIENLNKKVDDGFLDIWTYNAELLVLLENERTLDYHDSNVKNLYEKVRSQLKNNAKEIGNGCFEFYHKCDNTCMESVKNGTYDYPKYSEEAKLNREEIDGVKLESTRIYQILAIYSTVASSLVLVVSLGAISFWMCSNGSLQCRICI

SEQ ID NO: 17 - protein, 469 aa, Influenza virus
MNPNQKIITIGSVCMTIGMANLILQIGNIISIWISHSIQLGNQNQIETCNQSVITYENNTWVNQTYVNISNTNFAAGQSVVSVKLAGNSSLCPVSGWAIYSKDNSVRIGSKGDVFVIREPFISCSPLECRTFFLTQGALLNDKHSNGTIKDRSPYRTLMSCPIGEVPSPYNSRFESVAWSASACHDGINWLTIGISGPDNGAVAVLKYNGIITDTIKSWRNNILRTQESECACVNGSCFTVMTDGPSNGQASYKIFRIEKGKIVKSVEMNAPNYHYEECSCYPDSSEITCVCRDNWHGSNRPWVSFNQNLEYQIGYICSGIFGDNPRPNDKTGSCGPVSSNGANGVKGFSFKYGNGVWIGRTKSISSRNGFEMIWDPNGWTGTDNNFSIKQDIVGINEWSGYSGSFVQHPELTGLDCIRPCFWVELIRGRPKENTIWTSGSSISFCGVNSDTVGWSWPDGAELPFTIDK

SEQ ID NO: 18 - protein, 498 aa, Influenza virus
MASQGTKRSYEQMETGGERQDATEIRASVGRMIGGIGRFYIQMCTELKLSDYDGRLIQNSITIERMVLSAFDERRNKYLEEHPSAGKDPKKTGGPIYRRVDGKWMRELILYDKEEIRRVWRQANNGEDATAGLTHIMIWHSNLNDATYQRTRALVRTGMDPRMCSLMQGSTLPRRSGAAGAAVKGVGTIAMELIRMIKRGINDRNFWRGENGRRTRVAYERMCNILKGKFQTAAQRAMMDQVRESRNPGNAEIEDLIFLARSALILRGSVAHKSCLPACVYGLAVASGHDFEREGYSLVGIDPFKLLQNSQVVSLMRPNENPAHKSQLVWMACHSAAFEDLRVSSFIRGKKVIPRGKLSTRGVQIASNENVETMDSNTLELRSRYWAIRTRSGGNTNQQKASAGQISVQPTFSVQRNLPFERATVMAAFSGNNEGRTSDMRTEVIRMMESAKPEDLSFQGRGVFELSDEKATNPIVPSFDMSNEGSYFFGDNAEEYDS

SEQ ID NO: 19 - protein, 562 aa, Influenza virus
MAIIYLILLFTAVRGDQICIGYHANNSTEKVDTILERNVTVTHAKDILEKTHNGKLCKLNGIPPLELGDCSIAGWLLGNPECDRLLSVPEWSYIMEKENPRDGLCYPGSFNDYEELKHLLSSVKHFEKVKILPKDRWTQHTTTGGSRACAVSGNPSFFRNMVWLTKKGSNYPVAKGSYNNTSGEQMLIIWGVHHPNDETEQRTLYQNVGTYVSVGTSTLNKRSTPDIATRPKVNGLGSRMEFSWTLLDMWDTINFESTGNLIAPEYGFKISKRGSSGIMKTEGTLENCETKCQTPLGAINTTLPFHNVHPLTIGECPKYVKSEKLVLATGLRNVPQIESRGLFGAIAGFIEGGWQGMVDGWYGYHHSNDQGSGYAADKESTQKAFDGITNKVNSVIEKMNTQFEAVGKEFSNLERRLENLNKKMEDGFLDVWTYNAELLVLMENERTLDFHDSNVKNLYDKVRMQLRDNVKELGNGCFEFYHKCDDECMNSVKNGTYDYPKYEEESKLNRNEIKGVKLSSMGVYQILAIYATVAGSLSLAIMMAGISFWMCSNGSLQCRICI

(19) United States
(12) Patent Application Publication
(10) Pub. No.: US 2011/0159031 A1
(43) Pub. Date: Jun. 30, 2011

(54) Vaccine to Influenza A Virus

(75) Inventors: Falko G. Falkner, Orth/Donau (AT); Otfried Kistner, Vienna (AT); Annett Hessel, Orth/Donau (AT); P. Noel Barrett, Klosterneuburg/Weidling (AT); Hartmut Ehrlich, Vienna (AT); Georg Holzer, Wiener Neudorf (AT)

(73) Assignees: BAXTER INTERNATIONAL INC., Deerfield, IL (US); BAXTER HEALTHCARE S.A., Glattpark (Opfikon) (CH)

(21) Appl. No.: 12/976,145

(22) Filed: Dec. 22, 2010

Related U.S. Application Data
(60) Provisional application No. 61/289,309, filed on Dec. 22, 2009.

(51) Int. Cl. A61K 39/285; C12N 15/63; C12N 15/863; C12N 7/01; A61P 37/04; A61P 31/16

(52) U.S. Cl. 424/199.1; 435/320.1; 435/235.1

(57) ABSTRACT

The present invention relates, in general, to compositions and methods for administering a vaccine against influenza to a subject, the vaccine comprising a vaccinia virus vector and a hemagglutinin and neuraminidase gene, separate or in combination, from an influenza A virus.

### CROSS REFERENCE TO RELATED APPLICATIONS

This application claims the priority benefit of U.S. Provisional Patent Application No. 61/289,309, filed Dec. 22, 2009, hereby incorporated by reference in its entirety.

### FIELD OF THE INVENTION

The present invention relates, in general, to compositions and methods for administering a vaccine against influenza to a subject, the vaccine comprising a vaccinia virus vector and a hemagglutinin and neuraminidase gene, separate or in combination, from an influenza A virus.

### BACKGROUND OF THE INVENTION

The emergence and global spread of the new H1N1 influenza subtype in humans has made the development of vaccines against pandemic influenza viruses a global health priority. Several strategies are currently followed to produce pandemic vaccines, such as the development of inactivated whole virus vaccines, subunit vaccines, recombinant viral proteins and live vaccines. Vaccines based on inactivated influenza virus are usually derived from embryonated hens' eggs or, more recently, from permanent cell cultures. Protective immunity elicited by these vaccines is mainly based on neutralizing antibodies directed against the hemagglutinin gene (HA) (20, 21).

A broader immune response including efficient antibodies against the influenza surface proteins and induction of CD8 T cells, as induced by live vaccines, would be desirable. Attenuated live vaccines based on cold-adapted influenza strains (1, 10) or on nonreplicating, NS-1 gene-deleted influenza virus (12, 18) presumably have these advantages. Intranasal application of such pre-pandemic live vaccines, however, might result in new reassortant strains by co-infections in the respiratory tract with wild-type influenza strains raising safety concerns. In certain instances, influenza reassortants of the cold-adapted internal gene backbone with avian strains seem to have incompatible gene segments and induce only subpotent immune responses (6). Only the re-introduction of the polybasic cleavage site into the HA (previously deleted to attenuate the live virus) restored infectivity and immunogenicity (17). In another case, passaging of the live vaccine in host cells was required to achieve acceptable growth. Passaging, however, may result in reduced immunogenicity requiring screening of adequate reassortants (6). Further, the longterm effect of repeated intranasal administration of high doses of live vaccines on the olfactory system is largely unknown.

Live vaccines based on poxviral vectors, such as vaccinia virus vectors, including the highly inactivated modified vaccinia Ankara vector, are alternatives to prior vaccines as these vectors have a long safety record, induce T cell responses and are usually administered by demonstrated subcutaneous or intramuscular routes.

The purpose of this study was to evaluate the immune response and the protective potential of vaccinia-based influenza vaccines expressing the protective antigens hemagglutinin and neuraminidase, as exemplified against a newly identified H1N1 strain. Efficient induction of antibodies and surprisingly high levels of CD8 T cells were induced against both antigens.

### SUMMARY OF THE INVENTION

The present invention relates to the preparation and use of an antigenic composition or recombinant virus comprising a vaccinia virus vector and polynucleotides encoding influenza A genes that are relevant in producing an immune response and influenza infection. The antigenic composition and recombinant virus are useful as a vaccine to induce an immune response in a subject against the heterologous influenza genes expressed by the virus.

In one aspect, the invention provides an antigenic composition comprising a vaccinia virus vector comprising a polynucleotide encoding a hemagglutinin protein (HA) from influenza A and a polynucleotide encoding a neuraminidase protein (NA) from an influenza A virus, wherein the HA is of subtype selected from the group consisting of H1, H2, H3, H4, H5, H6, H7, H8, H9, H10, H11, H12, H13, H14, H15, and H16, and wherein the NA is of a subtype selected from the group consisting of N1, N2, N3, N4, N5, N6, N7, N8 and N9.

Exemplary influenza A subtypes contemplated by the invention include, any combination of H1 to H16 and N1 to N9, including H1N1, H2N1, H3N1, H4N1, H5N1, H6N1, H7N1, H8N1, H9N1, H10N1, H11N1, H12N1, H13N1, H14N1, H15N1, H16N1; H1N2, H2N2, H3N2, H4N2, H5N2, H6N2, H7N2, H8N2, H9N2, H10N2, H11N2, H12N2, H13N2, H14N2, H15N2, H16N2; H1N3, H2N3, H3N3, H4N3, H5N3, H6N3, H7N3, H8N3, H9N3, H10N3, H11N3, H12N3, H13N3, H14N3, H15N3, H16N3; H1N4, H2N4, H3N4, H4N4, H5N4, H6N4, H7N4, H8N4, H9N4, H10N4, H11N4, H12N4, H13N4, H14N4, H15N4, H16N4; H1N5, H2N5, H3N5, H4N5, H5N5, H6N5, H7N5, H8N5, H9N5, H10N5, H11N5, H12N5, H13N5, H14N5, H15N5, H16N5; H1N6, H2N6, H3N6, H4N6, H5N6, H6N6, H7N6, H8N6, H9N6, H10N6, H11N6, H12N6, H13N6, H14N6, H15N6, H16N6; H1N7, H2N7, H3N7, H4N7, H5N7, H6N7, H7N7, H8N7, H9N7, H10N7, H11N7, H12N7, H13N7, H14N7, H15N7, H16N7; H1N8, H2N8, H3N8, H4N8, H5N8, H6N8, H7N8, H8N8, H9N8, H10N8, H11N8, H12N8, H13N8, H14N8, H15N8, H16N8; H1N9, H2N9, H3N9, H4N9, H5N9, H6N9, H7N9, H8N9, H9N9, H10N9, H11N9, H12N9, H13N9, H14N9, H15N9, and H16N9. In some embodiments the influenza A subtype is a pandemic influenza A. Exemplary pandemic influenza subtypes include, but are not limited to, H1N1, H2N1, H3N2 and H5N1.

In one embodiment, the polynucleotide encoding the HA and the polynucleotide encoding the NA are derived from the same virus strain. In another embodiment, the polynucleotide encoding the HA and the polynucleotide encoding the NA are derived from different virus strains. In a related embodiment, the HA is derived from subtype H1 and the NA derived from subtype N1. In a further embodiment, the HA and NA are derived from influenza A strain virus A/California/07/2009.

In some embodiments, the H1 HA protein encoded by the polynucleotide is set out in FIG. 8 (SEQ ID NO: 14) or FIG. 14A (SEQ ID NO: 16). In a related embodiment, the N1 NA protein encoded by the polynucleotide is set out in FIG. 14B (SEQ ID NO: 17).

In certain embodiments, the HA is of subtype H2. In one embodiment, the amino acid sequence of the H2 protein is set out in FIG. 9 (SEQ ID NO: 15) or FIG. 15 (SEQ ID NO: 19).

It is further contemplated that the antigenic composition described herein optionally comprises a polynucleotide encoding an influenza A nucleoprotein (NP) protein. In one embodiment the amino acid sequence of the NP is set out FIG. 14C (SEQ ID NO: 18).

In some embodiments, the HA, NA and NP are derived from the same influenza strain or from different influenza A strains. For example, in some embodiments the antigenic composition comprises an HA, NA and NP from one or more of an H1N1, H2N1, H3N2 or H5N1 influenza virus.

In one embodiment, the invention contemplates an antigenic composition as described herein comprising polynucleotides encoding an H5, an N1 and an NP protein.

In certain embodiments, the vaccinia virus is selected from the group consisting of modified vaccinia Ankara (MVA), vaccinia virus Lister (VVL), and defective vaccinia Lister. Additional vaccinia viruses contemplated or use in the invention include, but are not limited to, MVA-575 (ECACC V00120707) and MVA-BN (ECACC V00083008).

In some embodiments, the antigenic composition described herein is characterized by the ability to propagate in vertebrate cell culture. In related embodiments, the vertebrate cell is selected from the group consisting of MRC-5, Vero, CV-1, MDCK, MDBK, HEK, H9, CEM, PerC6, BHK-21, BSC and LLC-MK2, DF-1, QT-35, or primary avian cells, such as primary chicken cells or chicken cell aggregates. In one embodiment, the vertebrate cell is a Vero cell.

In one embodiment, the antigenic composition further comprises a pharmaceutically acceptable carrier.

In a further embodiment, the HA of the antigenic composition comprises a polybasic cleavage site. In one embodiment, the polybasic cleavage site has the amino acid sequence RERRRKKR (SEQ ID NO: 1). It is contemplated that the HA may naturally express a polybasic cleavage site or may be altered to include a polybasic cleavage site. For example, strains H5 and H7 naturally comprise a polybasic cleavage site whereas H1 and other HA proteins do not naturally express a cleavage site but can be recombinantly engineered to contain a polybasic cleavage site.

In another aspect, the invention contemplates a recombinant vaccinia virus comprising a polynucleotide encoding a hemagglutinin protein (HA) from influenza A and a polynucleotide encoding a neuraminidase protein (NA) from an influenza A virus, wherein the HA is of subtype selected from the group consisting of H1, H2, H3, H4, H5, H6, H7, H8, H9, H10, H11, H12, H13, H14, H15, and H16, and wherein the NA is of a subtype selected from the group consisting of N1, N2, N3, N4, N5, N6, N7, N8 and N9. Any combination of the HA or NA subtype as described herein are useful in the recombinant virus.

Any combination of the HA or NA subtype as described herein is useful in the recombinant virus.

In one embodiment, the polynucleotide encoding the HA and the polynucleotide encoding the NA are derived from the same virus strain. In another embodiment, the polynucleotide encoding the HA and the polynucleotide encoding the NA are derived from different virus strains. In a related embodiment, the HA is derived from subtype H1 and the NA derived from subtype N1. In a further embodiment, the HA and NA are derived from influenza A strain virus A/California/07/2009.

In some embodiments, the H1 HA protein encoded by the polynucleotide is set out in FIG. 8 (SEQ ID NO: 14) or FIG. 14A (SEQ ID NO: 16). In a related embodiment, the N1 NA protein encoded by the polynucleotide is set out in FIG. 14B (SEQ ID NO: 17).

In certain embodiments, the HA is of subtype H2. In one embodiment, the amino acid sequence of the H2 protein is set out in FIG. 9 (SEQ ID NO: 15) or FIG. 15 (SEQ ID NO: 19).

It is further contemplated that the antigenic composition described herein optionally comprises a polynucleotide encoding an influenza A nucleoprotein (NP) protein. In one embodiment the amino acid sequence of the NP is set out FIG. 14C (SEQ ID NO: 18).

In some embodiments, the HA, NA and NP are derived from the same influenza strain or from different influenza strains. For example, in some embodiments the recombinant virus comprises an HA, NA and NP from one or more of an H1N1, H2N1, H3N2 or H5N1 influenza virus.

In one embodiment, the invention contemplates a recombinant virus as described herein comprising polynucleotides encoding an H5, an N1 and an NP protein.

In certain embodiments, the vaccinia virus is selected from the group consisting of modified vaccinia Ankara (MVA), vaccinia virus Lister (VVL), and defective vaccinia Lister. Additional vaccinia viruses contemplated or use in the invention include, but are not limited to, MVA-575 (ECACC V00120707) and MVA-BN (ECACC V00083008).

In some embodiments, the recombinant virus described herein is characterized by the ability to propagate in vertebrate cell culture. In related embodiments, the vertebrate cell is selected from the group consisting of MRC-5, Vero, CV-1, MDCK, MDBK, HEK, H9, CEM, PerC6, BHK-21, BSC and LLC-MK2, DF-1, QT-35, or primary avian cells, such as primary chicken cells or chicken cell aggregates. In one embodiment, the vertebrate cell is a Vero cell.

In one embodiment, the recombinant virus further comprises a pharmaceutically acceptable carrier.

In a further embodiment, the HA of the recombinant virus comprises a polybasic cleavage site. In one embodiment, the polybasic cleavage site has the amino acid sequence RERRRKKR (SEQ ID NO: 1). It is contemplated that the HA may naturally express a polybasic cleavage site or may be altered to include a polybasic cleavage site.

In a further aspect, the invention provides a vaccine comprising: a vaccinia virus vector comprising a polynucleotide encoding a hemagglutinin protein (HA) from influenza A and a polynucleotide encoding a neuraminidase protein (NA) from an influenza A virus, wherein the HA is from a subtype selected from the group consisting of H1, H2, H3, H4, H5, H6, H7, H8, H9, H10, H11, H12, H13, H14, H15, and H16, and wherein the NA is from a subtype selected from the group consisting of N1, N2, N3, N4, N5, N6, N7, N8 and N9, and wherein the polynucleotide encoding the HA and the polynucleotide encoding the NA are operatively linked to allow packaging of the polynucleotides into a virion.

In a related aspect, the invention contemplates a vaccine comprising, i) a first vaccinia virus vector comprising a polynucleotide encoding a hemagglutinin protein (HA) from influenza A, and ii) a second vaccinia vector comprising a polynucleotide encoding a neuraminidase protein (NA) from an influenza A virus, wherein the polynucleotide encoding the HA and the polynucleotide encoding the NA are operatively linked to allow packaging of the polynucleotides into a virion.

Any combination of the HA or NA subtype as described herein is useful in the vaccine.

In one embodiment, the polynucleotide encoding the HA and the polynucleotide encoding the NA are derived from the same virus strain. In another embodiment, the polynucleotide encoding the HA and the polynucleotide encoding the NA are derived from different virus strains. In a related embodiment, the HA is derived from subtype H1 and the NA derived from subtype N1. In a further embodiment, the HA and NA are derived from influenza A strain virus A/California/07/2009.

In some embodiments, the H1 HA protein encoded by the polynucleotide is set out in FIG. 8 (SEQ ID NO: 14) or FIG. 14A (SEQ ID NO: 16). In a related embodiment, the N1 NA protein encoded by the polynucleotide is set out in FIG. 14B (SEQ ID NO: 17).

In certain embodiments, the HA is of subtype H2. In one embodiment, the amino acid sequence of the H2 protein is set out in FIG. 9 (SEQ ID NO: 15) or FIG. 15 (SEQ ID NO: 19).

It is further contemplated that the vaccine described herein optionally comprises a polynucleotide encoding an influenza A nucleoprotein (NP) protein. In one embodiment the amino acid sequence of the NP is set out FIG. 14C (SEQ ID NO: 18).

In some embodiments, the HA, NA and NP are derived from the same influenza strain or from different influenza strains. For example, in some embodiments the antigenic composition comprises an HA, NA and NP from one or more of an H1N1, H2N1, H3N2 or H5N1 influenza.

In one embodiment, the invention contemplates a vaccine as described herein comprising polynucleotides encoding an H5, an N1 and an NP protein.

In certain embodiments, the vaccinia virus is selected from the group consisting of modified vaccinia Ankara (MVA), vaccinia virus Lister (VVL), and defective vaccinia Lister. Additional vaccinia viruses contemplated or use in the invention include, but are not limited to, MVA-575 (ECACC V00120707) and MVA-BN (ECACC V00083008).

In some embodiments, the vaccine described herein is characterized by the ability to propagate in vertebrate cell culture. In related embodiments, the vertebrate cell is selected from the group consisting of MRC-5, Vero, CV-1, MDCK, MDBK, HEK, H9, CEM, PerC6, BHK-21, BSC and LLC-MK2, DF-1, QT-35, or primary avian cells, such as primary chicken cells or chicken cell aggregates. In one embodiment, the vertebrate cell is a Vero cell.

In one embodiment, the vaccine further comprises a pharmaceutically acceptable carrier.

In a further embodiment, the HA of the vaccine comprises a polybasic cleavage site. In one embodiment, the polybasic cleavage site has the amino acid sequence RERRRKKR (SEQ ID NO: 1). It is contemplated that the HA may naturally express a polybasic cleavage site or may be altered to include a polybasic cleavage site.

In still another embodiment, it is contemplated that in the vaccine, the polynucleotide encoding the HA, the polynucleotide encoding the NA and the polynucleotide encoding the NP are each operably linked to a promoter. In related embodiments, the promoter is selected from the group consisting of a vaccinia mH5 promoter, a vaccinia early/late promoter, a bacteriophage T7 promoter, a thymidine kinase promoter, promoter of vaccinia virus gene coding for 7.5K polypeptide, a promoter of vaccinia virus gene coding for 19K polypeptide, a promoter of vaccinia virus gene coding for 42K polypeptide, a promoter of vaccinia virus gene coding for 28K polypeptide, a promoter of vaccinia virus gene coding for 11K polypeptide.

In certain embodiments, the vaccine further comprising an adjuvant.

In some embodiments, the vaccine is a live vaccine.

In certain embodiments, it is contemplated that the vaccine is administered in a dose having a TCID50 from at least 10² to at least 10¹⁰. In one embodiment, the TCID50 is from at least 10² to 10¹⁰, from at least 10⁴ to 10⁸, from at least 10⁶ to 10⁸ or from at least 10⁷ to 10⁹. In another embodiment, the TCID50 is at least 5×10⁷ or 5×10⁸. In still another embodiment, it is contemplated that a dose of the vaccine described herein exhibits a TCID50 of at least 10², 10³, 10⁴, 10⁵, 10⁶, 10⁷, 10⁸, or 10⁹. In one embodiment, the TCID50 is from 10⁶ to 10⁸. It is contemplated that the dose is administered in either a single dose or multiple doses. The total dose of vaccine may be split between multiple doses.

In an additional aspect, the invention provides a method for eliciting an immune response against at least one influenza virus strain in a subject, comprising administering an antigenic composition, a recombinant vaccinia virus or a vaccine as described herein in an amount effective to elicit the immune response against at least one influenza virus strain.

It is contemplated that an immune response includes, but is not limited to, antibodies against the influenza proteins as well as induction of influenza-specific T cell responses. Methods for measuring an immune response are described in greater detail in the Detailed Description and Examples.

In another aspect, the invention provides a method for preventing infection of a subject by an influenza virus comprising, administering to the subject an effective amount of an antigenic composition, a recombinant vaccinia virus or a vaccine as described herein in an amount effective to prevent infection of the subject by the influenza virus.

In one embodiment, the influenza strain is a pandemic strain. In another embodiment, the influenza strain is a seasonal influenza strain.

In certain embodiments, the subject is a vertebrate subject, including mammalian and avian subjects. In one embodiment, the subject is human.

The invention further provides, in a further aspect, a method of making a vaccine comprising a vaccinia virus vector and polynucleotide encoding a hemagglutinin protein (HA) from influenza A and a polynucleotide encoding a neuraminidase protein (NA) from an influenza A virus, wherein the HA is of subtype selected from the group consisting of H1, H2, H3, H4, H5, H6, H7, H8, H9, H10, H11, H12, H13, H14, H15, and H16, and wherein the NA is of a subtype selected from the group consisting of N1, N2, N3, N4, N5, N6, N7, N8 and N9, and optionally comprising a polynucleotide encoding an influenza A nucleoprotein (NP) protein, the method comprising transfecting the HA, NA, and optionally NP, polynucleotides into the virus in vertebrate cells under conditions suitable for growth of the virus.

Any combination of the HA or NA subtype as described herein are useful in the methods.

In certain embodiments, the method is characterized by viruses ability to propagate in vertebrate cell culture. In related embodiments, the vertebrate cell is selected from the group consisting of MRC-5, Vero, CV-1, MDCK, MDBK, HEK, H9, CEM, PerC6, BHK-21, BSC and LLC-MK2, DF-1, QT-35, or primary avian cells, such as primary chicken cells or chicken cell aggregates. In one embodiment, the vertebrate cell is a Vero cell.

In some embodiments, the vaccinia virus is selected from the group consisting of modified vaccinia Ankara (MVA), vaccinia virus Lister (VVL), and defective vaccinia Lister. Additional vaccinia viruses contemplated or use in the invention include, but are not limited to, MVA-575 (ECACC V00120707) and MVA-BN (ECACC V00083008).

In one embodiment, the polynucleotide encoding the HA and the polynucleotide encoding the NA are derived from the same virus strain. In another embodiment, the polynucleotide encoding the HA and the polynucleotide encoding the NA are derived from different virus strains. In a related embodiment, the HA is derived from subtype H1 and the NA derived from subtype N1. In a further embodiment, the HA and NA are derived from influenza A strain virus A/California/07/2009.

In some embodiments, the H1 HA protein encoded by the polynucleotide is set out in FIG. 8 (SEQ ID NO: 14) or FIG. 14A (SEQ ID NO: 16). In a related embodiment, the N1 NA protein encoded by the polynucleotide is set out in FIG. 14B (SEQ ID NO: 17).

In certain embodiments, the HA is of subtype H2. In one embodiment, the amino acid sequence of the H2 protein is set out in FIG. 9 (SEQ ID NO: 15) or FIG. 15 (SEQ ID NO: 19).

It is further contemplated that the method optionally comprises a polynucleotide encoding an influenza A nucleoprotein (NP) protein. In one embodiment the amino acid sequence of the NP is set out FIG. 14C (SEQ ID NO: 18).

In some embodiments, the HA, NA and NP are derived from the same influenza strain or from different influenza strains. For example, in some embodiments the methods of the invention comprise an HA, NA and NP from one or more of an H1N1, H2N1, H3N2 or H5N1 influenza.

In one embodiment, the methods contemplate a recombinant virus as described herein comprising polynucleotides encoding an H5, an N1 and an NP protein.

### BRIEF DESCRIPTION OF THE DRAWINGS

FIG. 1 illustrates expression of hemagglutinin and neuraminidase from the vaccinia virus vector. (A) Western blots of chicken cell (DF-1) lysates probed for hemagglutinin expression. Lane 1, marker size in kDa. Lane 2, formalin-inactivated purified H1N1 influenza virus. Lane 3, negative control lysate, replicating wt vaccinia virus. Lane 4, replicating virus rVVL-H1-CA, no trypsin treatment. Lane 5, replicating virus rVVL-H1-CA, with trypsin treatment. Lane 6, MVA-H1-CA, no trypsin treatment. Lane 7, MVA-H1-CA, with trypsin treatment. Lane 8, negative control lysate, MVA wt virus. Lane 9, uninfected cell lysate. HA0, unprocessed hemagglutinin; HA1 and HA2, the two processed HA subunits. (B) Western blot of DF-1 cell lysates probed for neuraminidase expression. Lanes 1-3, samples as above. Lane 4, replicating virus rVVL-N1-CA. Lane 5, MVA-N1-CA. Lane 6 and 7, negative controls, lysates of MVA wt virus and non-infected cells. The band around 75 kDa represents the neuramidase (NA).

FIG. 2 shows lung titers in Balb/c mice. The dots represent the lung titers of individual mice vaccinated with the different experimental vaccines or control preparations. Mice vaccinated with the controls (PBS, wild-type MVA (MVA wt) or Lister (VV-L wt) were not protected showing average log10 TCID50 titers of 5.2, 4.9 and 5.4, respectively. Mice vaccinated with inactivated vaccine (inactivated H1N1), or two different doses (10⁶, 10⁷ pfu) of MVA-H1-Ca or VV-L-H1-Ca were fully protected (86-100%). Partial protection was achieved with two dosages of the MVA neuraminidase viruses (MVA-N1-Ca), while full protection was seen with the Lister-based neuramindase virus (VV-L-N1-Ca). The dotted line represents the detection limit of log 10 2.21. Low titers in the range of log 10>2.21 to 3.0 were confirmed by a passage assay of the lung samples in MDCK cells. The numerical titers are shown in Table 3.

FIG. 3 shows the survival of SCID mice after passive transfer of influenza immune sera. (A) Groups of three mice were infected intranasally with doses of H1N1 wild-type virus in the range of 10² to 10⁵ TCID50 per animal and monitored over a 32 day period. The 10⁴ and 10⁵ doses were fully lethal. With the lower doses, mice survived the monitoring period. (B) Passive transfer of mouse sera. All SCID mice receiving the sera of Balb/c mice vaccinated with MVA-H1-Ca were protected, while the MVA-wt controls all died.

FIG. 4 shows T cell induction by the hemagglutinin constructs. (A) Frequencies of influenza antigen specific IFN-γ+ CD4 T-cells after immunizing two times with hemagglutinin constructs MVA-H1-CA (black bars), rVV-L-H1-CA (white bars) or inactivated vaccine (dotted bars) and stimulation with different antigens and peptides (shown on x-axis). Splenocytes were stimulated with protein antigens (formalin-inactivated monovalent bulk material) of the influenza strains H1N1 California (H1N1/CA), H1N1 Brisbane (H1N1/BR), H1N1 North Carolina (H1N1/NC), H5N1 Vietnam 1203 (H5N1/VN) and with peptide pools of overlapping 15-mer peptides of the swine flu hemagglutinin (H1/CA-PP) or neuraminidase (N1/CA-PP) antigens. (B) Frequencies of influenza hemagglutinin antigen specific IFN-γ+ CD8 T-cells after two dosages of vaccine. Splenocytes were stimulated with the peptide pools indicated above. The data are mean values (+/−SEM) of two independent experiments.

FIG. 5 shows T cell induction by neuraminidase constructs. (A) Frequencies of influenza antigen specific IFN-γ+ CD4 T-cells after immunizing two times with MVA-N1-CA (black bars), rVV-L-N1-CA (white bars) or inactivated vaccine (dotted bars) and stimulation with different antigens and peptides (shown on x-axis). Splenocytes were stimulated with protein antigens as described in FIG. 4. (B) Frequencies of influenza neuraminidase-specific IFN-γ+ CD8 T-cells after two dosages of vaccine. Splenocytes were stimulated with the peptide pools indicated in FIG. 4. The data are mean values (+/−SEM) of two independent experiments.

FIG. 6 illustrates T cell induction by the hemagglutinin expressing live vaccines in the lungs. Frequencies of influenza antigen specific IFN-γ+ CD4 T-cells (A, C) and CD8 T-cells (B, D) after immunizing two times with hemagglutinin constructs MVA-H1-Ca (A, B) or rVVL-H1-Ca (C, D) and stimulation with hemagglutinin (H1/CA-PP) or neuraminidase (N1/CA-PP) peptide pools. The filled (open) circles indicate lung (spleen) cells stimulated with the hemagglutinin peptide pool H1/CA PP. The filled (open) triangles indicate lung (spleen) cells stimulated with the neuraminidase peptide pool used as negative controls. Representative results of two independent experiments are shown.

FIG. 7 illustrates the modified HA cleavage site of the H1 of the A/California/07/2009(H1N1) strain (A) (SEQ ID NOs: 10 and 11) and the modified HA cleavage site of the H2 of the A/Singapore/1/57(H2N2) strain (B) (SEQ ID NOs: 12 and 13).

FIG. 8 shows the amino acid sequence of the modified H1 of the A/California/07/2009(H1N1) strain (SEQ ID NO: 14). The polybasic cleavage site is underlined.

FIG. 9 shows the amino acid sequence of the modified H2 of the A/Singapore/1/57(H2N2) strain (SEQ ID NO: 15). The polybasic cleavage site is underlined.

FIG. 10 is a schematic representation of the insertion of a HA-NA double gene cassette into the MVA genome using the D4R/D5R intergenic region as integration locus.

FIG. 11 is a schematic representation of the structure of the MVA-H1-N1-NP virus. The H1 and N1 gene cassette are inserted in the D4R/D5R intergenic region. The NP gene cassette is inserted in the del III region.

FIG. 12 shows a Western blot of infected CEC cell lysates probed for influenza antigens. A) Hemagglutinin expression. Lane 1, marker (sizes in kDa). Lane 2, formalin-inactivated purified H1N1 influenza virus. Lane 3, recombinant MVA-H1-Ca. Lane 4, recombinant MVA-H1-N1ca. Lane 5, negative control lysate infected with wt MVA. Lane 6, uninfected control lysate. HA0, unprocessed hemagglutinin; HA1 and HA2, the two processed HA subunits. B) Neuraminidase expression. Lanes 1-3, 5 and 6, samples as above. Lane 4, MVA-N1-Ca. NA, neuraminidase.

FIG. 13 illustrates lung titers of virus in Balb/c mice vaccinated with the MVA-H1-N1 virus. The dots represent the lung titers of individual mice vaccinated with the different doses (10⁶, 10⁵, 10⁴ pfu per animal) of MVA-H1-N1ca or control preparations. Mice vaccinated with the controls (wild-type MVA (MVA wt) or PBS) were not protected showing average log 10 TCID₅₀ titers of 5.3 or 4.7. Mice vaccinated with 10⁶ pfu of MVA-H1-N1-Ca were fully protected. Partial protection was achieved with 10⁴ and 10⁵ pfu MVA-H1-N1-Ca. The dotted line represents the detection limit of log 10, 2.21.

FIG. 14 shows the amino acid sequence of the wild-type of the A/California/07/2009 (H1N1) strain. (A) wild-type H1 (SEQ ID NO: 16); (B) wild-type N1 sequence (SEQ ID NO: 17); (C) wild-type NP sequence (SEQ ID NO: 18).

FIG. 15 shows the amino acid sequence of the wild-type H2 of the A/Singapore/1/57 (H2N2) strain (SEQ ID NO: 19). The single arginine used as cleavage is underlined.

### DETAILED DESCRIPTION

The present invention is directed to vaccines comprising hemagglutinin and neuraminidase genes of the influenza A subtype in a vaccinia virus vector. In some embodiments, the vaccine also comprises an influenza A nucleoprotein gene. The vaccine is useful to induce immunity to any influenza strain, and in certain aspects, is useful to induce protection to seasonal influenza, as well as pandemic influenza strains such as H1N1, H2N1, H5N1 and other pandemic strains. Vaccinia based influenza vaccines of the present invention provide improved protection compared to egg-based influenza vaccines since the vaccinia vector provides a more stable vector with reduced antigenic drift of the influenza genes, and provides improved activation of the humoral and cellular immune systems.

Unless otherwise defined, all technical and scientific terms used herein have the same meaning as commonly understood by one of ordinary skill in the art to which this invention belongs. The following references provide one of skill with a general definition of many of the terms used in this invention: Singleton, et al., DICTIONARY OF MICROBIOLOGY AND MOLECULAR BIOLOGY (2d ed. 1994); THE CAMBRIDGE DICTIONARY OF SCIENCE AND TECHNOLOGY (Walker ed., 1988); THE GLOSSARY OF GENETICS, 5TH ED., R. Rieger, et al. (eds.), Springer Verlag (1991); and Hale and Marham, THE HARPER COLLINS DICTIONARY OF BIOLOGY (1991).

Each publication, patent application, patent, and other reference cited herein is incorporated by reference in its entirety to the extent that it is not inconsistent with the present disclosure.

It is noted here that, as used in this specification and the appended claims, the singular forms “a,” “an,” and “the” include plural reference unless the context clearly dictates otherwise.

As used herein, the following terms have the meanings ascribed to them unless specified otherwise

The term “vaccinia virus vector” as used herein refers to a vaccinia virus genome useful as a vector to incorporate heterologous DNA, such as one or more polynucleotides encoding influenza A proteins, including but not limited to, the HA, NA, NP, M1 and M2 or PB1 proteins. The vaccinia virus vector also refers to the vaccinia virus particles which are assembled by the viral genome, and which comprises packaged proteins translated from the heterologous DNA as well as from homologous DNA. Exemplary vaccinia virus vectors useful in the invention include, but are not limited to, modified vaccinia Ankara (MVA), vaccinia virus Lister (VVL), defective vaccinia Lister, MVA-575 (ECACC V00120707), and MVA-BN (ECACC V00083008).

A “recombinant vaccinia virus” refers to a vaccinia virus particle comprising heterologous DNA, such as one or more polynucleotides encoding influenza A proteins, including but not limited to, the HA, NA, NP, M1, M2 or PB1 proteins. Accordingly, the term recombinant vaccinia virus is used interchangeably with aspects of the term vaccinia virus vector as the term vaccinia virus vector relates to vaccinia virus particles which are assembled by the viral genome, and which comprises packaged proteins translated from the heterologous DNA.

The term “derived from” is used to indicate a parental source for all or a portion of a polynucleotide or polypeptide sequence that is altered or mutated from a starting polynucleotide or polypeptide, including for example a wild-type or naturally-occurring polynucleotide or polypeptide sequence. In other words, a polynucleotide or polypeptide is derived from a parental wild type sequence which is altered in one or more bases or amino acids such that the resulting polynucleotide or polypeptide no longer has the same sequence as the parental wild-type sequence.

The term “subtype” as used herein refers to the different groupings of influenza A strains that can be divided and classified based on the HA and NA genes that are expressed in the virus strain. The influenza A subtype nomenclature is based on the HA subtype, e.g., the subtype is any one of the 16 different HA genes known in the art, and the NA subtype, e.g., any of the 9 different NA genes known in the art. Exemplary subtypes, include but are not limited to, H5N1, H1N1, H3N2, and many more known in the art.

The term “strain” as used herein refers to the particular virus variant of a given species, e.g., influenza A or B species, and subtype in an influenza A virus. For example, the virus A/California/7/2009 is an Influenza A virus, subtype H1N1, with the strain name A/California/7/2009.

The term “antigenic composition” refers to a composition comprising material which stimulates the immune system and elicits an immune response in a host or subject. The term “elicit an immune response” refers to the stimulation of immune cells in vivo in response to a stimulus, such as an antigen. The immune response consists of both cellular immune response, e.g., T cell and macrophage stimulation, and humoral immune response, e.g., B cell and complement stimulation and antibody production. The cellular and humoral immune response are not mutually exclusive, and it is contemplated that one or both are stimulated by an antigenic composition, virus or vaccine as described herein. Immune response may be measured using techniques well-known in the art, including, but not limited to, antibody immunoassays, proliferation assays, and others described in greater detail in the Detailed Description and Examples.

The term “attenuated” is used to describe a virus or antigenic composition which demonstrates reduced virulence (compared to a wild-type virus). Attenuated virus is typically but not always administered intranasally.

The term “inactivated” is used herein to describe a virus that is also known in the art as a “killed” or “dead” virus. An inactivated virus is a whole virus without virulent properties and is produced from a “live” virus, regardless of whether the virus has been previously attenuated in any manner. Inactivated virus is typically, but not always, administered via intramuscular injection. Administration of an inactivated virus is contemplated via any route described herein.

The term “vaccine” as used herein refers to a composition comprising a recombinant virus as described herein, which is useful to establish immunity to the virus in the subject. It is contemplated that the vaccine comprises a pharmaceutically acceptable carrier and/or an adjuvant. It is contemplated that vaccines are prophylactic or therapeutic. A “prophylactic” treatment is a treatment administered to a subject who does not exhibit signs of a disease or exhibits only early signs for the purpose of decreasing the risk of developing pathology. The compounds of the invention may be given as a prophylactic treatment to reduce the likelihood of developing a pathology or to minimize the severity of the pathology, if developed. A “therapeutic” treatment is a treatment administered to a subject who exhibits signs or symptoms of pathology for the purpose of diminishing or eliminating those signs or symptoms. The signs or symptoms may be biochemical, cellular, histological, functional, subjective or objective.

The term “live vaccine” as used herein refers to a vaccine comprising a live virus, which are typically attenuated in some manner, but that retain their immunogenic properties. A live virus can infect cells, but can be either a replication-deficient live virus, such that it cannot replicate and produce additional viral particles, or replication-competent virus.

A “fragment” of a polypeptide refers to any portion of the polypeptide smaller than the full-length polypeptide or protein expression product. Fragments are, in one aspect, deletion analogs of the full-length polypeptide wherein one or more amino acid residues have been removed from the amino terminus and/or the carboxy terminus of the full-length polypeptide. Accordingly, “fragments” are a subset of deletion analogs described below.

An “analogue,” “analog” or “derivative,” which are used interchangeably, refers to a compound, e.g., a peptide or polypeptide, substantially similar in structure and having the same biological activity, albeit in certain instances to a differing degree, to a naturally-occurring molecule. Analogs differ in the composition of their amino acid sequences compared to the naturally-occurring polypeptide from which the analog is derived, based on one or more mutations involving (i) deletion of one or more amino acid residues at one or more termini of the polypeptide and/or one or more internal regions of the naturally-occurring polypeptide sequence, (ii) insertion or addition of one or more amino acids at one or more termini (typically an “addition” analog) of the polypeptide and/or one or more internal regions (typically an “insertion” analog) of the naturally-occurring polypeptide sequence or (iii) substitution of one or more amino acids for other amino acids in the naturally-occurring polypeptide sequence. It is contemplated that a recombinant virus of the invention comprises an analog of a viral gene, including any one or more than one of an HA, NA, PB1, PB2, PA, M (M1 and M2), NS (NS1 and NS2) and NP gene.

In one aspect, an analog exhibits about 70% sequence similarity but less than 100% sequence similarity with the wild-type or naturally-occurring sequence, e.g., a peptide. Such analogs or derivatives are, in one aspect, comprised of non-naturally occurring amino acid residues, including by way of example and not limitation, homoarginine, ornithine, penicillamine, and norvaline, as well as naturally occurring amino acid residues. Such analogs or derivatives are, in another aspect, composed of one or a plurality of D-amino acid residues, or contain non-peptide interlinkages between two or more amino acid residues. In one embodiment, the analog or derivative may be a fragment of a polypeptide, wherein the fragment is substantially homologous (i.e., at least 70%, at least 75%, at least 80%, at least 85%, at least 90%, or at least 95% homologous) over a length of at least 5, 10, 15, 20, 25, 30, 35, 40, 45 or 50 amino acids of the wild-type polypeptide.

Substitutions are conservative or non-conservative based on the physico-chemical or functional relatedness of the amino acid that is being replaced and the amino acid replacing it. Substitutions of this type are well known in the art. Alternatively, the invention embraces substitutions that are also non-conservative. Exemplary conservative substitutions are described in Lehninger, [Biochemistry, 2nd Edition; Worth Publishers, Inc., New York (1975), pp. 71-77] and set out below.

CONSERVATIVE SUBSTITUTIONS

| SIDE CHAIN | |
|---|---|
| CHARACTERISTIC | AMINO ACID |
| Non-polar (hydrophobic): | |
| A. Aliphatic | A L I V P |
| B. Aromatic | F W |
| C. Sulfur-containing | M |
| D. Borderline | G |
| Uncharged-polar: | |
| A. Hydroxyl | S T Y |
| B. Amides | N Q |
| C. Sulfhydryl | C |
| D. Borderline | G |
| Positively charged (basic) | K R H |
| Negatively charged (acidic) | D E |

Alternatively, exemplary conservative substitutions are set out immediately below.

CONSERVATIVE SUBSTITUTIONS II

| | EXEMPLARY |
|---|---|
| ORIGINAL RESIDUE | SUBSTITUTION |
| Ala (A) | Val, Leu, Ile |
| Arg (R) | Lys, Gln, Asn |
| Asn (N) | Gln, His, Lys, Arg |
| Asp (D) | Glu |
| Cys (C) | Ser |
| Gln (Q) | Asn |
| Glu (E) | Asp |
| His (H) | Asn, Gln, Lys, Arg |
| Ile (I) | Leu, Val, Met, Ala, Phe, |
| Leu (L) | Ile, Val, Met, Ala, Phe |
| Lys (K) | Arg, Gln, Asn |
| Met (M) | Leu, Phe, Ile |
| Phe (F) | Leu, Val, Ile, Ala |
| Pro (P) | Gly |
| Ser (S) | Thr |
| Thr (T) | Ser |
| Trp (W) | Tyr |
| Tyr (Y) | Trp, Phe, Thr, Ser |
| Val (V) | Ile, Leu, Met, Phe, Ala |

The term “isolated” as used herein refers to a virus or antigenic composition that is removed from its native environment. Thus, an isolated biological material is free of some or all cellular components, i.e., components of the cells in which the native material occurs naturally (e.g., cytoplasmic or membrane component). In one aspect, a virus or antigenic composition is deemed isolated if it is present in a cell extract or supernatant. In the case of nucleic acid molecules, an isolated nucleic acid includes a PCR product, an isolated mRNA, a cDNA, or a restriction fragment.

The term “purified” as used herein refers to a virus or antigenic composition that has been isolated under conditions that reduce or eliminate the presence of unrelated materials, i.e., contaminants, including endogenous materials from which the composition is obtained. By way of example, and without limitation, a purified virion is substantially free of host cell or culture components, including tissue culture or egg proteins and non-specific pathogens. In various embodiments, purified material substantially free of contaminants is at least 50% pure; at least 60%, at least 70%, at least 80%, at least 90%, at least 95%, at least 96%, at least 97%, at least 98% or even at least 99% pure. Purity can be evaluated by chromatography, gel electrophoresis, immunoassay, composition analysis, biological assay, and other methods known in the art.

The term “pharmaceutical composition” refers to a composition suitable for administration to a subject animal, including humans and mammals. A pharmaceutical composition comprises a pharmacologically effective amount of a virus or antigenic composition of the invention and also comprises a pharmaceutically acceptable carrier. A pharmaceutical composition encompasses a composition comprising the active ingredient(s), and the inert ingredient(s) that make up the pharmaceutically acceptable carrier, as well as any product which results, directly or indirectly, from combination, complexation or aggregation of any two or more of the ingredients. Accordingly, the pharmaceutical compositions of the present invention encompass any composition made by admixing a compound or conjugate of the present invention and a pharmaceutically acceptable carrier.

The term “pharmaceutically acceptable carrier” include any and all clinically useful solvents, dispersion media, coatings, antibacterial and antifungal agents, isotonic and absorption delaying agents, buffers, and excipients, such as a phosphate buffered saline solution, 5% aqueous solution of dextrose or mannitol, and emulsions, such as an oil/water or water/oil emulsion, and various types of wetting agents and/or adjuvants. Suitable pharmaceutical carriers and formulations are described in Remington's Pharmaceutical Sciences, 19th Ed. (Mack Publishing Co., Easton, 1995). Pharmaceutical carriers useful for the composition depend upon the intended mode of administration of the active agent. Typical modes of administration include, but are not limited to, enteral (e.g., oral) or parenteral (e.g., subcutaneous, intramuscular, intravenous or intraperitoneal injection; or topical, transdermal, or transmucosal administration). A “pharmaceutically acceptable salt” is a salt that can be formulated into a compound or conjugate for pharmaceutical use including, e.g., metal salts (sodium, potassium, magnesium, calcium, etc.) and salts of ammonia or organic amines.

The term “pharmaceutically acceptable” or “pharmacologically acceptable” refers to a material which is not biologically or otherwise undesirable, i.e., the material may be administered to an individual without causing any undesirable biological effects or interacting in a deleterious manner with any of the components of the composition in which it is contained, or when administered using routes well-known in the art, as described below.

### Influenza Genes

Influenza viruses are segmented negative-strand RNA viruses and belong to the Orthomyxoviridae family. Influenza A virus consists of nine structural proteins and codes additionally for one nonstructural NS1 protein with regulatory functions. The influenza virus segmented genome contains eight negative-sense RNA (nsRNA) gene segments (PB2, PB1, PA, NP, M, NS, HA and NA) that encode at least ten polypeptides, including RNA-directed RNA polymerase proteins (PB2, PB1 and PA), nucleoprotein (NP), neuraminidase (NA), hemagglutinin (subunits HA1 and HA2), the matrix proteins (M1 and M2) and the non-structural proteins (NS1 and NS2) (Krug et al., In The Influenza Viruses, R. M. Krug, ed., Plenum Press, N.Y., 1989, pp. 89 152).

Influenza virus ability to cause widespread disease is due to its ability to evade the immune system by undergoing antigenic change, which is believed to occur when a host is infected simultaneously with both an animal influenza virus and a human influenza virus. During mutation and reassortment in the host, the virus may incorporate an HA and/or NA surface protein gene from another virus into its genome, thereby producing a new influenza subtype and evading the immune system.

### Hemagglutinin

HA is a viral surface glycoprotein comprising approximately 560 amino acids and representing 25% of the total virus protein. It is responsible for adhesion of the viral particle to, and its penetration into, a host cell in the early stages of infection. There are 16 known HA subtypes, categorized as an H1, H2, H3, H4, H5, H6, H7, H8, H9, H10, H11, H12, H13, H14, H15 or H16 subtype.

Cleavage of the virus HA0 precursor into the HA1 and HA2 subfragments is a necessary step in order for the virus to infect a cell. Thus, cleavage is required in order to convert new virus particles in the host cells into virions capable of infecting new cells. Cleavage is known to occur during transport of the integral HA0 membrane protein from the endoplasmic reticulum of the infected cell to the plasma membrane. In the course of transport, hemagglutinin undergoes a series of co- and post-translational modifications including proteolytic cleavage of the precursor HA into the amino-terminal fragment HA1 and the carboxy terminal HA2. One of the primary difficulties in growing influenza strains in primary tissue culture or established cell lines arises from the requirement for proteolytic cleavage activation of the influenza hemagglutinin in the host cell.

Although it is known that an uncleaved HA can mediate attachment of the virus to its neuraminic acid-containing receptors on a cell surface, it is not capable of the next step in the infectious cycle, which is fusion. It has been reported that exposure of the hydrophobic amino terminus of the HA2 by cleavage is required so that it can be inserted into the target cell, thereby forming a bridge between virus and target cell membrane. This process is followed by fusion of the two membranes and entry of the virus into the target cell.

Proteolytic activation of HA involves cleavage at an arginine residue by a trypsin-like endoprotease, which is often an intracellular enzyme that is calcium dependent and has a neutral pH optimum. Since the activating proteases are cellular enzymes, the infected cell type determines whether the HA is cleaved. The HA of the mammalian influenza viruses and the nonpathogenic avian influenza viruses are susceptible to proteolytic cleavage only in a restricted number of cell types. On the other hand, HA of pathogenic avian viruses among the H5 and H7 subtypes are cleaved by proteases present in a broad range of different host cells. Thus, there are differences in host range resulting from differences in hemagglutinin cleavability which are correlated with the pathogenic properties of the virus.

The differences in cleavability are due to differences in the amino acid sequence of the cleavage site of the HA. Sequence analyses show that the HA1 and HA2 fragments of the HA molecule of the non-pathogenic avian and all mammalian influenza viruses are linked by a single arginine. In contrast, the pathogenic avian strains have a sequence of several basic amino acids at the cleavage site with the common denominator being lysine-arginine or arginine-arginine, e.g., RRRK (see e.g., SEQ ID NO: 1). H5 and H7 subtypes exhibit the polybasic cleavage sites. The hemagglutinins of all influenza viruses are cleaved by the same general mechanism resulting in the elimination of the basic amino acids.

In some embodiments, in HA genes that do not exhibit a polybasic cleavage site, a polybasic cleavage site is inserted into the gene in order to induce cleavage of the HA0 protein into the HA1 and HA2 proteins. In certain embodiments, the cleavage site has the sequence RERRRKKR (SEQ ID NO: 1).

### Neuraminidase

Neuraminidase is a second membrane glycoprotein of the influenza A viruses. The presence of viral NA has been shown to be important for generating a multi-faceted protective immune response against an infecting virus. NA is a 413 amino acid protein encoded by a gene of 1413 nucleotides. Nine different NA subtypes have been identified in influenza viruses (N1, N2, N3, N4, N5, N6, N7, N8 and N9), all of which have been found among wild birds. NA is involved in the destruction of the cellular receptor for the viral HA by cleaving terminal neuraminic acid (also called sialic acid) residues from carbohydrate moieties on the surfaces of infected cells. NA also cleaves sialic acid residues from viral proteins, preventing aggregation of viruses. Using this mechanism, it is hypothesized that NA facilitates release of viral progeny by preventing newly formed viral particles from accumulating along the cell membrane, as well as by promoting transportation of the virus through the mucus present on the mucosal surface. NA is an important antigenic determinant that is subject to antigenic variation.

Administration of chemical inhibitors of neuraminidase limits the severity and spread of viral infections. Neuraminidase inhibitors combat influenza infection by preventing the virus from budding from the host cell. Exemplary NA inhibitors include, but are not limited to, zanamivir, administered by inhalation; oseltamivir, administered orally; and peramivir administered parenterally.

### Internal Genes of Influenza

In addition to the surface proteins HA and NA, influenza virus comprises six additional internal genes, which give rise to eight different proteins, including polymerase genes PB1, PB2 and PA, matrix proteins M1 and M2, nucleoprotein (NP), and non-structural proteins NS1 and NS2 (Horimoto et al., Clin Microbiol Rev. 14(1):129-49, 2001).

In order to be packaged into progeny virions, viral RNA is transported from the nucleus as a ribonucleoprotein complex composed of the three influenza virus polymerase proteins, the nucleoprotein (NP), and the viral RNA, in association with the influenza virus matrix 1 (M1) protein and nuclear export protein (Marsh et al., J Virol, 82:2295-2304, 2008). The M1 protein that lies within the envelope is thought to function in assembly and budding.

A limited number of M2 proteins are integrated into the virions (Zebedee, J. Virol. 62:2762-2772, 1988). They form tetramers having H+ ion channel activity, and, when activated by the low pH in endosomes, acidify the inside of the virion, facilitating its uncoating (Pinto et al., Cell 69:517-528, 1992). Amantadine is an anti-influenza drug that prevents viral infection by interfering with M2 ion channel activity, thus inhibiting virus uncoating.

NS1 protein, a nonstructural protein, has multiple functions, including regulation of splicing and nuclear export of cellular mRNAs as well as stimulation of translation. The major function of NS1 seems to be to counteract the interferon activity of the host, since an NS1 knockout virus was viable although it grew less efficiently than the parent virus in interferon-nondefective cells (Garcia-Sastre, Virology 252:324-330, 1998).

NS2 protein has been detected in virus particles. The average number of NS2 proteins in a virus particle was estimated to be 130-200 molecules. An in vitro binding assay shows direct protein-protein contact between M1 and NS2. NS2-M1 complexes were also detected by immunoprecipitation in virus-infected cell lysates (Yasuda et al., Virology 196:249-55, 1993). The NS2 protein, known to exist in virions (Richardson et al., Arch. Virol. 116:69-80, 1991; Yasuda et al., Virology 196:249-255, 1993), is thought to play a role in the export of RNP from the nucleus through interaction with M1 protein (Ward et al., Arch. Virol. 140:2067-2073, 1995).

### Influenza Genes in Vaccinia Vectors

Pandemics of influenza emerge from the aquatic bird reservoir, adapt to humans, modify their severity, and cause seasonal influenza. The catastrophic Spanish H1N1 virus may have obtained all of its eight gene segments from the avian reservoir, whereas the Asian H2N2 and the Hong Kong H3N2 pandemics emerged by reassortment between the circulating human virus and an avian H2 or H3 donor. Of the 16 hemagglutinin subtypes, the H2, H5, H6, H7, and H9 viruses are considered to have pandemic potential (32).

The major antigen of influenza virus is the hemagglutinin located on the surface of the virion. It induces the majority of neutralizing antibodies. Therefore, in standard inactivated vaccines, only the HA is quantified and dosing is based on HA content.

The neuraminidase (NA) is the second most important antigen followed by the nucleoprotein (NP). The NA induces antibodies that inhibit neuraminidase activity of the NA molecule, required for efficient release of the virus from the cell. Inhibition of NA function, and therefore release of the virus from infected cells, means reduction of virus titer.

The nucleoprotein (NP) is the type antigen (the A antigen, of the influenza A type viruses) and is highly conserved in influenza virus. This antigen contains dominant T cell epitopes including CD8 T cell epitopes (21).

In one embodiment, influenza genes of the influenza A subtypes are useful in the methods and compositions of the invention. For example, influenza A virus having any HA subtype is contemplated, including any of the H1 to H16 subtypes, excluding the H5 subtype. In a still further embodiment it is contemplated that an influenza virus having any of NA subtypes N1 to N9 is useful for the invention. In various embodiments, the influenza genes are derived from either a seasonal influenza strain or a pandemic influenza strain.

In certain embodiments, it is contemplated that when generating a recombinant vaccinia virus, antigenic composition or vaccine of the invention, the HA and NA subtype are derived from different strains. In other embodiments, it is contemplated that when generating a recombinant vaccinia virus, antigenic composition or vaccine of the invention, the HA and NA subtype are derived from the same strain, and optionally the NP is derived from the same strain of influenza virus. Exemplary combinations include, but are not limited to, e.g., H1 and N1 insertions having H1 and N1 genes from the same or different H1N1 virus strains, or H2 and N1 inserts having H2 and N1 genes from the same or different H2N1 virus strains.

It is further contemplated that any of the following influenza A subtypes are useful in the invention: H1N1, H2N1, H3N1, H4N1, H5N1, H6N1, H7N1, H7N1, H8N1, H9N1, H10N1, H11N1, H12N1, H13N1, H14N1, H15N1, H16N1; H1N2, H2N2, H3N2, H4N2, H5N2, H6N2, H7N2, H8N2, H9N2, H10N2, H11N2, H12N2, H13N2, H14N2, H15N2, H16N2; H1N3, H2N3, H3N3, H4N3, H5N3, H6N3, H7N3, H8N3, H9N3, H10N3, H11N3, H12N3, H13N3, H14N3, H15N3, H16N3; H1N4, H2N4, H3N4, H4N4, H5N4, H6N4, H7N4, H8N4, H9N4, H10N4, H11N4, H12N4, H13N4, H14N4, H15N4, H16N4; H1N5, H2N5, H3N5, H4N5, H5N5, H6N5, H7N5, H8N5, H9N5, H10N5, H11N5, H12N5, H13N5, H14N5, H15N5, H16N5; H1N6, H2N6, H3N6, H4N6, H5N6, H6N6, H7N6, H8N6, H9N6, H10N6, H11N6, H12N6, H13N6, H14N6, H15N6, H16N6; H1N7, H2N7, H3N7, H4N7, H5N7, H6N7, H7N7, H8N7, H9N7, H10N7, H11N7, H12N7, H13N7, H14N7, H15N7, H16N7; H1N8, H2N8, H3N8, H4N8, H5N8, H6N8, H7N8, H8N8, H9N8, H10N8, H11N8, H12N8, H13N8, H14N8, H15N8, H16N8; H1N9, H2N9, H3N9, H4N9, H5N9, H6N9, H7N9, H8N9, H9N9, H10N9, H11N9, H12N9, H13N9, H14N9, H15N9, and H16N9. Influenza A viruses of the following subtypes have been identified previously, H1N1, H2N2, H1N2, H3N2, H3N8, H4N6, H5N1, H5N2, H5N3, H5N9, H6N1, H6N2, H6N5, H7N1, H7N7, H8N4, H9N2, H10N7, H11N6, H12N5, H13N6, H14N5, H15N8, H15N9, H16N3. Table 1 lists exemplary HA and NA genes from influenza A strains useful for generating a recombinant virus, antigenic composition or vaccine as described herein.

TABLE 1

| Gene | Accession | |
|---|---|---|
| Subtype | No. | |
| | | Hemagglutinin Genes |
| H1 | NC_002017 | Influenza A virus (A/Puerto Rico/8/34(H1N1)) segment 4, complete sequence |
| H1 | FJ966952 | Influenza A virus (A/California/05/2009(H1N1)) segment 4 hemagglutinin (HA) gene, complete cds. |
| H2 | L20410 | Influenza A virus (A/Singapore/1/1957(H2N2)) hemagglutinin (HA)gene, complete cds. |
| H2 | L11126 | Influenza A virus (A/Berlin/3/64 (H2N2)) hemagglutinin (HA) gene, complete cds. |
| H3 | CY050836 | Influenza A virus (A/New York/3487/2009(H3N2)) segment 4, complete sequence. |
| H3 | CY008628 | Influenza A virus (A/Canterbury/257/2005(H3N2)) segment 4, complete sequence. |
| H4 | M25290 | Influenza A virus (A/turkey/Minnesota/833/1980(H4N2)) hemagglutinin (HA) gene, complete cds. |
| H4 | FJ428583 | Influenza A virus (A/mallard/Poyang Lake/15/2007(H4N6)) segment 4 hemagglutinin (HA) gene, complete cds. |
| H5 | EF541403 | Influenza A virus (A/Viet Nam/1203/2004(H5N1)) segment 4 hemagglutinin (HA) gene, complete cds. |
| H5 | AF082035 | Influenza A virus (A/Chicken/Hong Kong/786/97 (H5N1)) hemagglutinin H5 mRNA, complete cds. |
| H6 | GQ117282 | Influenza A virus (A/ring-billed gull/GA/421733/2001 (H6N4)) segment 4 hemagglutinin (HA) gene, complete cds. |
| H6 | CY045343 | Influenza A virus (A/northern shoveler/California/K138/2005(H6N2)) segment 4, complete sequence. |
| H7 | EF576989 | Influenza A virus (A/duck/AB/AFLBs8734c16/2007(H7)) segment 4, complete sequence. |
| H7 | AY240925 | Influenza A virus (A/avian/NY/73063-6/00(H7N2)) hemagglutinin (HA) gene, complete cds. |
| H8 | CY043848 | Influenza A virus (A/mallard/Netherlands/1/2006(H8N4)) segment 4, complete sequence. |
| H8 | AB450435 | Influenza A virus (A/duck/Alaska/702/1991(H8N7)) HA gene for haemagglutinin, complete cds. |
| H9 | GU071984 | Influenza A virus (A/chicken/Iran/THLBM868/2007(H9N2)) segment 4 hemagglutinin (HA) gene, complete cds. |
| H9 | CY023992 | Influenza A virus (A/duck/Shantou/3577/2003(H9N2)) segment 4 sequence. |
| H10 | EU124207 | Influenza A virus (A/Duck/Indonesia/Jakarta Utara1631-29/2006(H10)) segment 4 hemagglutinin (HA) gene, complete cds. |
| H10 | M21647 | Influenza A virus (A/chicken/Germany/N/1949(H10N7)) hemagglutinin precursor, gene, complete cds. |
| H11 | CY021437 | Influenza A virus (A/environment/Delaware/235/2005(H11N6)) segment 4, complete sequence. |
| H11 | D90306 | Influenza A virus (A/duck/England/1/1956(H11N6)) gene for hemagglutinin precursor, complete cds. |
| H12 | CY021877 | Influenza A virus (A/mallard/Maryland/1131/2005(H12N5)) segment 4, complete sequence. |
| H12 | AB288334 | Influenza A virus (A/duck/Alberta/60/1976(H12N5)) HA gene for haemagglutinin, complete cds. |
| H13 | EU835900 | Influenza A virus (A/gull/Astrakhan/1818/1998(H13N6)) hemagglutinin (HA) gene, complete cds. |
| H13 | AB292664 | Influenza A virus (A/gull/Maryland/704/1977(H13N6)) HA gene for haemagglutinin, complete cds. |
| H14 | FJ975075 | Influenza A virus (A/herring gull/Astrakhan/267/1982(H14N5)) segment 4 hemagglutinin (HA) gene, complete cds. |
| H14 | AM922165 | Influenza A virus (A/mallard/Gur/263/82(H14N3)) partial HA gene for hemagglutinin, genomic RNA. |
| H15 | L43917 | Influenza A virus (A/shearwater/West Australia/2576/79(H15N9)) hemagglutinin mRNA, complete cds. |
| H15 | CY006032 | Influenza A virus (A/Australian shelduck/Western Australia/1756/1983(H15N2)) segment 4, complete sequence. |
| H16 | EU148600 | Influenza A virus (A/mallard/Gurjev/785/83(H16N3)) hemagglutinin precursor (HA) gene, complete cds. |
| H16 | EU564109 | Influenza A virus (A/Fulica atra/Volga/635/1986(H16N3)) segment 4 hemagglutinin (HA) gene, complete cds. |
| | | Neuraminidase Genes |
| N1 | FJ969517 | Influenza A virus (A/California/04/2009(H1N1)) segment 6 neuraminidase (NA) gene, complete cds. |
| N1 | CY030233 | Influenza A virus (A/Brisbane/59/2007(H1N1)) segment 6 sequence. |
| N2 | EU199420 | Influenza A virus (A/Brisbane/10/2007(H3N2)) segment 6 neuraminidase (NA) gene, complete cds. |
| N2 | GU052277 | Influenza A virus (A/turkey/England/1969(H3N2)) segment 6 neuraminidase (NA) gene, complete cds. |
| N3 | GU052285 | Influenza A virus (A/seal/Massachusetts/3911/1992(H3N3)) segment 6 neuraminidase (NA) gene, complete cds. |
| N3 | GU052831 | Influenza A virus (A/environment/California/508249/2007(H5N3)) segment 6 neuraminidase (NA) gene, complete cds. |
| N4 | CY039550 | Influenza A virus (A/northern shoveler/California/AKS273/2007(H8N4)) segment 6 sequence. |
| N4 | EU557563 | Influenza A virus (A/northern pintail/Alaska/44204-158/2006(H6N4)) segment 6 neuraminidase (NA) gene, complete cds. |
| N5 | EU871915 | Influenza A virus (A/mallard/MN/105/2000(H5N5)) segment 6 neuraminidase (NA) gene, complete cds. |
| N5 | CY033334 | Influenza A virus (A/northern shoveler/California/HKWF1046/2007(H3N5)) segment 6 sequence. |
| N6 | GU051165 | Influenza A virus (A/ruddy turnstone/New Jersey/950/2005(H3N6)) segment 6 neuraminidase (NA) gene, complete cds. |
| N6 | GU053454 | Influenza A virus (A/mallard/Ohio/684/2002(H4N6)) segment 6 neuraminidase (NA) gene, complete cds. |
| N7 | FJ517261 | Influenza A virus (A/shorebird/DE/1346/2001 (H5N7)) segment 6 neuraminidase (NA) gene, complete cds. |
| N7 | GU051509 | Influenza A virus (A/mallard/Minnesota/17/1999(H7N7)) segment 6 neuraminidase (NA) gene, complete cds. |
| N8 | CY015091 | Influenza A virus (A/turkey/Ireland/1378/1983(H5N8)) segment 6, complete sequence. |
| N8 | CY043810 | Influenza A virus (A/ring-necked duck/California/K90/2005(H6N8)) segment 6, complete sequence. |
| N9 | AB292782 | Influenza A virus (A/duck/Hong Kong/562/1979(H10N9)) NA gene for neuraminidase, complete cds. |
| N9 | GU053360 | Influenza A virus (A/blue-winged teal/Ohio/467/2001 (H11N9)) segment 6 neuraminidase (NA) gene, complete cds. |

Additional influenza A viruses contemplated include, but are not limited to, A/Brisbane/59/2007 (H1N1); A/Brisbane/10/2007 (H3N2); A/Solomon Islands/3/2006 (H1N1); A/Uruguay/716/2007; A/Wisconsin/67/2005 (H3N2); A/New Caledonia/20/99(H1N1); A/California/7/2004(H3N2); A/New York/55/2004; A/Wellington/1/2004(H3N2); A/Fujian/411/2002(H3N2); A/Moscow/10/99(H3N2); A/Panama/2007/99; A/Sydney/5/97 (H3N2); A/Beijing/262/95 (H1N1); and viruses having like properties to any of the above viruses. U.S. Patent Publication No. 20090010962, incorporated herein by reference, describes influenza A H1N1 viruses useful in the invention.

A list of identified Influenza A strains, including influenza A H1N1 strains is available from the World Health Organization (WHO) and United States Centers for Disease Control (CDC) databases of Influenza A subtypes. The National Center for Biotechnology Information (NCBI) database maintained by the United States National Library of Medicine also maintains an updated database describing the length and sequence of HA and NA genes of identified viruses of influenza A species. Strains listed by these organizations and viral strains described in other commercial and academic databases, or in literature publications and known in the art, are contemplated for use in the invention. It is also contemplated that additional influenza A strains hereafter identified and isolated are also useful in the invention. Accordingly, any strain specifically exemplified in the specification and those known or after discovered in the art are amenable to the recombinant vaccinia virus, antigenic composition, vaccine and methods of the invention.

In certain embodiments, a recombinant live vaccine minimally contains the HA molecule. Due to its dominant role, expression of HA is sufficient for vaccine function. In related embodiments, to induce a broader immune response (concerning cross protection and functional antibody function), the NA gene is inserted into the vaccinia vector.

Optionally, the NP gene is inserted into the vaccinia vector comprising any of the above-mentioned HA and/or NA genes. The NP is minimally protective on its own, however, it induces efficient CD8 T cell responses that are important for clearance of virus from infected cells and thus contribute to a positive clinical course of the infection. A live vaccine containing HA, NA and NP provides broad protection and can induce cross protection to additional influenza viruses.

### Vaccinia Vectors

Vaccinia viruses belong to the family of poxviruses. A modified vaccinia Ankara virus (MVA) was obtained by mutation and selection of the original vaccinia virus Ankara after 575 passages in chicken embryo fibroblast cultures. The safety of this MVA is reflected by biological, chemical and physical characteristics. MVA has a reduced molecular weight, six primary deletions in the genome (approximately 31 kB, or 10% of its genome), and is highly attenuated for most mammalian cells, i.e. DNA and protein is synthesized but essentially no viable viral particles are produced due to a limited ability to replicate efficiently in primate cells. Despite its limited replication, MVA has been shown to be a highly effective expression vector (Sutter et al., Proc. Natl. Acad. Sci. USA 89:10847-10851, 1992), raising protective immune responses in primates for parainfluenza virus (Durbin et al. J. Infect. Dis. 179:1345-1351, 1999), measles (Stittelaar et al. J. Virol. 74:4236-4243, 2000), and immunodeficiency viruses (Barouch et al., J. Virol. 75:5151-5158, 2001; Ourmanov et al., J. Virol. 74:2740-2751, 2000). The relatively high immunogenicity of MVA has been attributed in part to the loss of several viral anti-immune defense genes (Blanchard et al., J. Gen. Virol. 79:1159-1167, 1998). See also US Patent Publication No. 20070048861. The modified vaccinia virus Ankara developed by Anton Mayr was deposited at the European Collection of Cell Cultures (ECACC), Salisbury, UK, under depository No. V 94012707. Additional methods and modified vaccinia viruses are described in U.S. Pat. Nos. 5,185,146, 6,682,743, and US Patent Publication No. 20090311746.

The genomic organization of the MVA genome has been described in Antoine et al., (Virology 244:365-396, 1998). The 178 kb genome of MVA comprises 193 individual open reading frames (ORFs), which code for proteins of at least 63 amino acids in length. In comparison with the highly infectious Variola virus and also the prototype of Vaccinia virus, namely the strain Copenhagen, many ORFs of MVA are fragmented or truncated (Antoine et al., supra). However, nearly all ORFs, including the fragmented and truncated ORFs, get transcribed and translated into proteins. U.S. Patent Publ. No. 20090311746 describes new insertion sites for insertion of heterologous DNA into the MVA genome, which are located in the intergenic regions (IGRs) of the viral genome, which are, in turn, located between or are flanked by two adjacent open reading frames (ORFs) of the MVA genome.

In some embodiments, influenza genes are inserted into nonreplicating poxviral vectors and used as live vaccines. An exemplary vaccinia strain used is the modified vaccinia Ankara strain. Another nonreplicating vaccinia vector is the defective vaccinia virus growing only in complementing cell lines (28) that may be used alternatively to MVA. Expression of the H5 hemagglutinin and protection of animals after lethal challenge with virulent H5N1 viruses has been recently shown in this system (29).

MVA are well known and available in the art. MVA strains useful for the present invention include, but are not limited to, MVA-575 (deposited at the European Collection of Animal Cell Cultures under the deposition number ECACC V00120707), MVA-BN (ECACC V00083008). In some embodiments, the MVA isolate is obtained from the National Institutes of Health (Bethesda, Md.), MVA1974NIH clone 1, an isolate of 1974 that has no history of spongiform encephalopathies.

Methods to introduce exogenous DNA sequences by a plasmid vector into a vaccinia genome and methods to obtain recombinant vaccinia are well known to the person skilled in the art and, additionally, are deducible from the following references: Molecular Cloning, A laboratory Manual. 2^{nd} Edition. Sambrook, et al., Cold Spring Harbor Laboratory Press. (1989); Virology Methods Manual. Mahy et al., Academic Press. (1996); Molecular Virology: A Practical Approach. Davison and Elliott. IRL Press at Oxford University Press. Oxford (1993). Chapter 9—Expression of genes by Vaccinia virus vectors; Current Protocols in Molecular Biology. John Wiley and Son Inc. (1998). Chapter 16, section IV: Expression of proteins in mammalian cells using Vaccinia viral vector.

The inserted DNA can be linear or circular, e.g., a plasmid. It is contemplated that the DNA contains at least one partial sequence from a non-essential segment of the vaccinia virus DNA. Non-essential segments of the vaccinia virus DNA are known to those skilled in the art. One example of a non-essential segment of this type is the thymidine kinase gene and its adjacent regions (Weir et al., J. Virol. 46:530-537, 1983).

The heterologous DNA can be introduced into the cells by means of homologous recombination using a plasmid construct. The plasmid is transfected into host cell, for example by means of calcium phosphate precipitation, electroporation, liposomes, microinjection, or by other methods known to those skilled in the art.

In certain embodiments, the vaccinia vector further comprises at least one marker or selection gene. Selection genes transduce a particular resistance to a cell, whereby a certain selection method becomes possible. Selection genes are well-known to one of ordinary skill in the art, including but not limited to, neomycin resistance gene (NPT) or phosphoribosyl transferase gene (gpt).

Marker genes induce a color reaction in transduced cells, which can be used to identify transduced cells. The skilled practitioner is familiar with a variety of marker genes, which can be used in a poxviral system, including but not limited to, β-Galactosidase (β-gal), β-Glucosidase (β-glu), Green Fluorescence protein (GFP) or Blue Fluorescence Protein.

In still a further embodiment, the exogenous DNA sequence comprises a spacer sequence, which separates a poxviral transcription control element and/or coding sequence in the exogenous DNA sequence from the stop codon and/or the start codon of the adjacent ORFs. This spacer sequence between the stop/start codon of the adjacent ORF and the inserted coding sequence in the exogenous DNA has the advantage to stabilize the inserted exogenous DNA and, thus, any resulting recombinant virus. The size of the spacer sequence is variable as long as the sequence is without own coding or regulatory function.

### Cells Lines

Vertebrate cell lines are useful for culture and growth of vaccinia virus including MVA. Exemplary vertebrate cells useful to culture virus for the preparation of vaccine include, but are not limited to: MRC-5, MRC-9, Vero and CV-1 (African Green monkey): HEK (human embryonic kidney), PerC6 (human retinoblast); BHK-21 cells (baby hamster kidney), BSC (monkey kidney cell) and LLC-MK2 (monkey kidney), avian cell lines including DF-1, QT-35 and primary avian cell cultures, such as primary chicken cells, chicken cell aggregates.

Vero cells are an accepted cell line for production of vaccine according to the World Health Organization. In one embodiment, viruses of the present invention are grown in Vero cells as described in the Examples below.

### Vaccines

It is contemplated that desired influenza A genes from a virus strain are used to produce a recombinant vaccinia virus vaccine that leads to increased immune response to the influenza viral proteins. Many types of viral vaccines are known, including but not limited to, attenuated, inactivated, subunit, and split vaccines.

Attenuated vaccines are live viral vaccines that have been altered in some manner to reduce pathogenicity and no longer cause disease. Attenuated viruses are produced in several ways, including growth in tissue culture for repeated generations and genetic manipulation to mutate or remove genes involved in pathogenicity. For example, in one embodiment, viral genes and/or proteins identified as involved in pathogenicity or involved in the disease manifestation, are mutated or changed such that the virus is still able to infect and replicate within a cell, but it cannot cause disease. Attenuation of virus has also been successful by insertion of a foreign epitope into a viral gene segment or by deletion of genome segments necessary for viral replication, via recombinant methods or serial passage in cell culture, resulting in a replication deficient virus.

Subunit vaccines are killed vaccines. Production of subunit vaccine involves isolating a portion of the virus that activates the immune system. In the case of influenza, subunit vaccines have been prepared using purified HA and NA, but any mixture of viral proteins is used to produce a subunit vaccine. Generally, the viral protein, such as HA is extracted from recombinant virus forms and the subunit vaccine is formulated to contain a mixture of these viral proteins from different strains.

In certain embodiments, a vaccine as described herein is prepared using standard adjuvants and vaccine preparations known in the art. Adjuvants include, but are not limited to, saponin, non-ionic detergents, vegetable oil, mineral gels such as aluminum hydroxide, surface active substances such as lysolecithin, pluronic polyols, polyanions, peptides, oil or hydrocarbon emulsions, keyhole limpet hemocyanins, and potentially useful human adjuvants such as N-acetyl-muramyl-L-threonyl-D-isoglutamine (thr-MDP), N-acetyl-nor-muramyl-L-alanyl-D-isoglutamine, N-acetylmuramyl-L-alanyl-D-isoglutaminyl-L-alanine-2-(1′-2′-dipalmitoyl-s-n-glycero-3-hydroxyphosphoryloxy)-ethylamine, BCG (bacille Calmette-Guerin) Corynebacterium parvum, ISCOMs, nano-beads, squalene, and block copolymers, which are contemplated for use alone or in combination.

ISCOM is an acronym for Immune Stimulating Complex, described initially in Morein et al. (Nature 308:457-460,1984). ISCOM's are a novel vaccine delivery system and are unlike conventional adjuvants. An ISCOM is formed in two ways. In some embodiments, the antigen is physically incorporated in the structure during its formulation. In other embodiments, an ISCOM-matrix (as supplied by, for example, Isconova) does not contain antigen but is mixed with the antigen of choice by the end-user prior to immunization. After mixing, the antigens are present in solution with the ISCOM-matrix but are not physically incorporated into the structure.

In one embodiment, the adjuvant is an oil in water emulsion. Oil in water emulsions are well known in the art, and have been suggested to be useful as adjuvant compositions (EP 399843; WO 95/17210, U.S. Patent Publication No. 20080014217). In one embodiment, the metabolizable oil is present in an amount of 0.5% to 20% (final concentration) of the total volume of the antigenic composition or isolated virus, at an amount of 1.0% to 10% of the total volume, or in an amount of 2.0% to 6.0% of the total volume.

In some embodiments, oil-in-water emulsion systems useful as adjuvant have a small oil droplet size. In certain embodiments, the droplet sizes will be in the range 120 to 750 nm, or from 120 to 600 nm in diameter.

In order for any oil in water composition to be suitable for human administration, the oil phase of the emulsion system comprises a metabolizable oil. The oil may be any vegetable oil, fish oil, animal oil or synthetic oil, which is not toxic to the recipient and is capable of being transformed by metabolism. Nuts, seeds, and grains are common sources of vegetable oils. Synthetic oils are also part of this invention and can include commercially available oils such as NEOBEE® and others. A particularly suitable metabolizable oil is squalene. Squalene (2,6,10,15,19,23-Hexamethyl-2,6,10,14,18,22-tetracosahexaene) is an unsaturated oil which is found in large quantities in shark-liver oil, and in lower quantities in olive oil, wheat germ oil, rice bran oil, and yeast, and is a particularly suitable oil for use in this invention. Squalene is a metabolizable oil by virtue of the fact that it is an intermediate in the biosynthesis of cholesterol (Merck index, 10th Edition, entry no. 8619). Exemplary oils useful for an oil in water emulsion, include, but are not limited to, sterols, tocols, and alpha-tocopherol.

In additional embodiments, immune system stimulants are added to the vaccine and/or pharmaceutical composition. Immune stimulants include: cytokines, growth factors, chemokines, supernatants from cell cultures of lymphocytes, monocytes, or cells from lymphoid organs, cell preparations and/or extracts from plants, cell preparation and, or extracts from bacteria (e.g., BCG, mycobacterium, Corynebacterium), parasites, or mitogens, and novel nucleic acids derived from other viruses, or other sources (e.g. double stranded RNA, CpG) block co-polymers, nano-beads, or other compounds known in the art, used alone or in combination.

Particular examples of adjuvants and other immune stimulants include, but are not limited to, lysolecithin; glycosides (e.g., saponin and saponin derivatives such as Quil A (QS7 and QS21) or GPI-0100); cationic surfactants (e.g. DDA); quaternary hydrocarbon ammonium halogenides; pluronic polyols; polyanions and polyatomic ions; polyacrylic acids, non-ionic block polymers (e.g., Pluronic F-127); and 3D-MPL (3 de-O-acylated monophosphoryl lipid A). See e.g., U.S. Patent Publication Nos. 20080187546 and 20080014217.

### Immunoassays

Various techniques are known in the art for detecting immunospecific binding of an antibody to an antigen which are useful to detect the antigenicity and induction of an immune response of a recombinant virus, antigenic composition or vaccine of the present invention. An early method of detecting interaction between an antigen and an antibody involved detection and analysis of the complex by precipitation in gels. A further method of detecting an antigen-antibody binding pair includes the use of radioiodinated detector antibodies or a radioiodinated protein which is reactive with IgG, such as Protein A. These early methods are well known to persons skilled in the art, as reviewed in Methods in Enzymology 70:166-198, 1980.

Serological assays are widely used in the determination of influenza diagnosis as well as in research studies regarding the epidemiology and antigenicity of viral strains. In particular, the hemagglutinin inhibition (HI or HAI) assay is widely used (Meisner et al., J Virol. 82:5079-83, 2008; Couch et al., Vaccine. 25:7656-63, 2007). The HI assay is also useful to show the antigenicity of the modified HA molecule, and assist in the characterization of the modified HA protein as more or less antigenic than a non-modified HA protein.

The HI assay determines the ability of antibodies from a serum sample to bind with a standardized reference. In the HI assay, serial dilutions (titers) of serum sample are mixed with standard amounts of erythrocytes and their association into complexes is detected visually. The lowest level of titered serum that results in a visible complex is the assay result.

A single radial diffusion (SRD) assay was developed by Wood et al. (J Biol Standardization 5:237-47, 1997) which determines the level of HA antigen in a sample. The SRD assay compares the zone of diffusion sites of a reference antigen and a test antigen (e.g., a vaccine) when the antigen are bound by HA-specific antibodies.

Detection of antigen specific T cell responses are also contemplated to analyze the antigenicity of the modified virus vectors and the vaccines described herein. In one embodiment, induction of CD4 and/or CD8 T cells are measured. The levels of active T cells are analyzed in organs or tissues of the subject, including, but not limited to, lung, spleen, and lymph node. Additionally, it is contemplated that levels of cytokines secreted by T cells are analyzed to detect antigen specific T cell activation. In one embodiment, the cytokine is interferon-γ (IFN-γ). In an exemplary embodiment, the levels of antigen specific T cell activation is reduced in subjects receiving a vaccine described herein. Methods of measuring the above parameters are carried out using etechnicques well-known in the art, including, but not limited to, cell proliferation assays, FACS analysis of cytokine or cell levels, and ELISA or ELISPOT methods. In another exemplary embodiment, levels of activated CD4 cells and/or activated CD8 cells are reduced in subjects receiving a vaccine described herein. In a further embodiment, levels of IFN-γ are reduced in subjects receiving a vaccine described herein.

### Pharmaceutical Formulations and Administration

The administration of the vaccine composition is generally for prophylactic purposes. The prophylactic administration of the composition serves to prevent or attenuate any subsequent infection. A “pharmacologically acceptable” composition is one tolerated by a recipient patient. It is contemplated that an effective amount of the vaccine is administered. An “effective amount” is an amount sufficient to achieve a desired biological effect such as to induce enough humoral or cellular immunity. This may be dependent upon the type of vaccine, the age, sex, health, and weight of the recipient. Examples of desired biological effects include, but are not limited to, production of no symptoms, reduction in symptoms, reduction in virus titer in tissues or nasal secretions, complete protection against infection by influenza virus, and partial protection against infection by influenza virus.

A vaccine or composition of the present invention is physiologically significant if its presence results in a detectable change in the physiology of a recipient patient that enhances at least one primary or secondary humoral or cellular immune response against at least one strain of an infectious influenza virus. The vaccine composition is administered to protect against viral infection. The “protection” need not be absolute, i.e., the influenza infection need not be totally prevented or eradicated, if there is a statistically significant improvement compared with a control population or set of patients. Protection may be limited to reducing the severity or rapidity of onset of symptoms of the influenza virus infection.

In one embodiment, an attenuated or inactivated vaccine composition of the present invention is provided either before the onset of infection (so as to prevent or attenuate an anticipated infection) or after the initiation of an actual infection, and thereby protects against viral infection.

In one aspect, methods of the invention include a step of administration of a pharmaceutical composition. The virus, antigenic composition or vaccine is administered in any means known in the art, including via inhalation, intranasally, orally, and parenterally. Examples of parental routes of administration include intradermal, intramuscular, intravenous, intraperitoneal and subcutaneous administration.

In one embodiment, influenza vaccine administration is based on the viral titer of the vaccine sample. In some embodiments, the dose of vaccine is based on the viral TCID50, e.g., the median tissue culture infective dose; the amount of a pathogenic agent that will produce pathological change in 50% of cell cultures inoculated. It is contemplated that the TCID50 is from 10² to 10¹⁰, from 10⁴ to 10⁸, from 10⁶ to 10⁸ or from 10⁷ to 10⁹. In another embodiment, the TCID50 is 5×10⁷ or 5×10⁸. In still another embodiment, it is contemplated that a dose of the vaccine described herein exhibits a TCID50 of at least 10², 10³, 10⁴, 10⁵, 10⁶, 10⁷, 10⁸, or 10⁹. It is contemplate that the dose is administered in either a single dose or multiple doses. The total dose of vaccine may be split between multiple doses.

In a related embodiment, if a replication competent vaccinia is used, a vaccine composition of the present invention comprises from about 10² to 10⁹ plaque forming units (PFU)/ml, or any range or value therein, where the virus is attenuated. In some embodiments, the vaccine composition comprises about 10², about 10³, about 10⁴, about 10⁵, about 10⁶, about 10⁷, about 10⁸ or about 10⁹ PFU/ml. It is further contemplated that the vaccine composition comprises from 10² to about 10⁴ PFU/ml, from about 10⁴ to about 10⁶ PFU/ml, or from about 10⁶ to about 10⁹ PFU/ml.

It is contemplated that, in some embodiments, the dose of vaccine is adjusted based on the adjuvant used for vaccine preparation.

Accordingly, it is contemplated that single vaccine dosages include those having a TCID50 as described herein and are provided in single or multiple dosages at the same or different amount of viral titer.

When administered as a solution, the vaccine is prepared in the form of an aqueous solution. Such formulations are known the art, and are prepared by dissolution of the antigen and other appropriate additives in the appropriate solvent. Such solvents include water, saline, ethanol, ethylene glycol, and glycerol, for example. Suitable additives include certified dyes and antimicrobial preservatives, such as thimerosal (sodium ethylmercuithiosalicylate). Such solutions may be stabilized using standard methods, for example, by addition of partially hydrolyzed gelatin, sorbitol, or cell culture medium and may be buffered using standard methods, using, for example reagents such as sodium hydrogen phosphate, sodium dihydrogen, phosphate, potassium hydrogen phosphate and/or potassium dihydrogen phosphate or TRIS. Liquid formulations may also include suspensions and emulsions. The preparation of suspensions include, for example, using a colloid mill, and emulsions include for example using a homogenizer.

In one embodiment, it is contemplated that the vaccine carrier is a polymeric delayed release system. Synthetic polymers are useful in the formulation of a vaccine to effect the controlled release of antigens using well-known techniques in the art.

### Kits

The invention also contemplates kits for administering the antigenic composition, recombinant virus or vaccine as described herein packaged in a manner which facilitates their use to practice methods of the invention. In one embodiment, such a kit includes a compound or composition described herein, packaged in a container such as a sealed bottle or vessel, with a label affixed to the container or included in the package that describes use of the compound or composition in practicing the method. Preferably, the compound or composition is packaged in a unit dosage form. The kit may further include a device suitable for administering the composition according to a specific route of administration or for practicing a screening assay. Preferably, the kit contains a label that describes use of the composition. In some embodiments, the kit comprises instructions for administering the composition to a human subject.

Additional aspects and details of the invention will be apparent from the following examples, which are intended to be illustrative rather than limiting.

### EXAMPLES

### Example 1

### Construction and Characterization of Recombinant Vaccinia Comprising Influenza HA and NA Genes

In order to generate a vaccine against the H1N1 viral proteins from a pandemic influenza A strain, H1 and N1 genes were isolated and inserted into a vaccinia virus vector and the immunogenicity of the vaccine tested in vivo in mice, including the ability of the vaccine to induce a humoral response in vivo.

Materials and Methods

Animals: All animal experiments were reviewed by the Institutional Animal Care and Use Committee (IACUC) and approved by the Austrian regulatory authorities. All animal experiments were conducted in accordance with Austrian laws on animal experimentation and guidelines set out by the Association for Assessment and Accreditation of Laboratory Animal Care International (AAALAC). Animals were housed according to AAALAC guidelines, in housing facilities accredited by the AAALAC.

Cells and viruses: The Vero (CCL-81) and DF-1 (CCL-12203) cell lines were obtained from the American Type Culture Collection. They were cultivated in DMEM (Biochrom AG) containing 5% fetal calf serum (FCS). Chicken embryo cells (CEC) were cultivated in M199 (GIBCO, Inc./Invitrogen Inc., Carlsbad, Calif.) containing 5% fetal calf serum (FCS). Madin-Darby canine kidney (MDCK) cells were maintained serum free in ULTRA-MDCK medium (Bio Whittaker, Walkersville, Md.).

The influenza virus A/California/7/2009 (H1N1; CDC #2009712112) was kindly provided by the Centers for Disease Control and Prevention (CDC, Atlanta, USA).

The vaccinia virus strain Lister/Elstree (VR-862) was obtained from the American Type Culture Collection. The basis of the Lister constructs was the subcloned virus vpDW-862/Elstree. The MVA strain (MVA 1974/NIH clone 1) was kindly provided by B. Moss (National Institutes of Health).

Cloning and sequencing of hemagglutinin and neuraminidase genes: The hemagglutinin (Genbank #FJ966082) and neuraminidase (Genbank #FJ966084) sequences of A/California/4/2009 were synthesized (Geneart, Regensburg, Germany). Both synthetic genes are driven by the strong early/late vaccinia virus promoter mH5 and terminate with a vaccinia virus specific stop signal downstream of the coding region that is absent internally. Both expression cassettes were cloned into the MVA transfer plasmid pHA-vA (2) resulting in pHA-mH5-H1-Ca and pHA-mH5-N1-Ca, respectively. The insertion plasmids direct the gene cassettes into the MVA HA-locus, close to deletion III of MVA (2). Both gene cassettes were inserted in parallel into the vaccinia transfer plasmid pER-mH5-PL resulting in pER-mH5-H1-Ca or pER-mH5-N1-Ca, respectively. The plasmid pER-mH5-PL was obtained by insertion of the vaccinia virus promoter mH5, a vaccinia virus stop signal (TTTTTNT) and a multiple cloning site (StuI, NcoI, PvuII, SpeI, HindIII, SacI, XmaI, SalI, NotI) into plasmid pER (5). For the MVA insertion plasmids, the gene cassettes were cloned into pHA-vA (14) resulting in pHA-mH5-H1-Ca and pHA-mH5-N1-Ca, respectively.

Construction of recombinant vaccinia viruses: VV-L-H1-CA and VV-L-N1-CA. Twenty micrograms of pER-mH5-H1-Ca or pER-mH5-N1-Ca plasmid DNA were transfected into vaccinia virus Lister infected Vero cells by calcium phosphate precipitation and further processed as described previously (5). Plaque isolates were purified three times and expanded for large scale preparations in Vero cells. The vaccinia virus stocks were prepared in Vero cells by infection with 0.1 MOI for 72 hours. Infected cells were harvested and sucrose cushion purified viral stocks were prepared (Holzer et al., Virology 249(1): 160-6, 1998) MVA-H1-CA and MVA-N1-CA. Twenty micrograms of pHA-mH5-H1-Ca or pHA-mH5-N1-Ca plasmid DNA were transfected into MVA infected CEC by calcium phosphate precipitation and further processed as described previously (14). The purified recombinant virus isolates were expanded for large scale preparations in CEC and purified by centrifugation through sucrose cushion (Joklik, W., Virology 18:9-18, 1962).

Western blot analysis: Expression of the H1 or N1 proteins by recombinant vaccinia virus or MVA was detected by Western blotting. Vero cells in case of the VV-L constructs, or the avian cell line DF-1 (4) in case of MVA, were infected at a multiplicity of infection of 0.1 for 48 h. MVA-H1-CA or VVL-H1-CA infected cells were harvested by scraping or by adding trypsin. MVA-N1-Ca or VVL-N1-Ca infected cells were harvested by scraping. Sonicated cell lysates were loaded onto 12% polyacrylamide gels (BioRad, Inc, Hercules, Calif.) and afterwards blotted on nitrocellulose membrane (Invitrogen, Inc, Carlsbad, Calif.). To detect H1 protein, a sheep antiserum against the A/California/7/2009 hemagglutinin (NIBSC 09/152) was used. Donkey-anti-sheep alkaline phosphatase-conjugated IgG (Sigma Inc., St. Louis, Mo.) was used as a secondary antibody. To detect N1 protein, a polyclonal rabbit anti avian influenza A neuraminidase (Abcam, ab21304, Cambridge, Mass.) was used. Goat-anti-rabbit alkaline phosphatase-conjugated IgG (Sigma Inc.) was used as a secondary antibody. A whole virus vaccine H1N1 A/California/7/2009 (8) served as positive control.

Immunizations of Balb/c mice for analysis of cellular and humoral immunity: Groups of 6 female Balb/c mice (8-10 weeks old) were immunized intramuscularly either once (day 0) or twice (days 0 and 21) with 10⁶ or 10⁷ pfu of recombinant MVA-H1-Ca or VV-L-H1-Ca. Control groups were immunized with 10⁷ pfu wild-type MVA, 10⁷ pfu wild type VV-L, 3.75 μg of whole virus vaccine H1N1 A/California/7/2009 or with PBS buffer. To analyze the vaccines effects on cellular immunity, blood samples were taken for IgG subclass and HI titer determinations on days 7 and 27 and spleens were obtained for IFN-γ analyses at days 8 and 28 post-immunization after euthanizing the mice. To analyze induction of humoral immunity, blood samples were taken for IgG subclass and HI titer determinations on days 20 and 41. Mice were challenged intranasally with 10⁵ TCID50/ml of A/California/7/2009 on day 42 and lungs were removed three days later and frozen at ≦60° C.

Immunizations of Balb/c mice for analysis of cellular immunity: Groups of 5 female Balb/c mice (8-10 weeks old) were immunized intramuscularly on days 0 and 21 with 10⁶ pfu of recombinant MVA-H1-Ca or rVVL-H1-Ca, wild-type MVA or VV-L, 3.75 mg of whole virus vaccine H1N1 A/California/7/2009 (inactivated H1N1) or with PBS buffer. Spleens were obtained for IFN-γ analyses at day 28 post-immunization. Furthermore, groups of 5 mice immunized two times with 10⁶ pfu of recombinant MVA-H1-Ca, rVVLH1-Ca, or MVA wt were challenged intranasally with 10⁵ TCID₅₀ per animal of A/California/7/2009 on day 42, and lungs and spleens were collected after euthanizing the mice 7 days after the booster immunization (day 28), at the time of challenge (day 41) or three days thereafter (day 45) for determination of influenza-specific T-cell frequencies.

Preparation of the lung samples and titration: Mice were euthanized and lungs were removed on day 3 post challenge with wild-type H1N1 virus. These tissue samples were stored at <−60° C. until they were transferred into homogenization tubes (PRECELLYS® Ceramic Kit 2.8 mm, PEQLAB Biotechnologie GmbH, Germany) containing 1 ml cell medium supplemented with antibiotics. The lungs were homogenized two times at 5000 rpm for twenty seconds with 5 seconds pause between the intervals with a tissue homogenizer (PRECELLYS24®, PEQLAB Biotechnologie GmbH). The infectious H1N1 virus titer in homogenized lung samples was determined by a TCID50 assay performed by titration on Madin-Darby canine kidney (MDCK) cells by serial ten-fold dilutions of samples as described (9).

Passage assay of low-titer lung samples in MDCK cells: Lung samples with titers <3 log 10 TCID₅₀ were verified by this passage assay. Confluent MDCK cells (grown in 75 cm² Roux flasks) were infected with 100 μl of mouse lung sample. The adsorption medium was 10 ml ULTRA-MDCK™ medium (BioWhittaker®) and contained 1 μg/ml TPCK trypsin (Sigma Inc.). After one hour virus adsorption 40 ml ULTRA-MDCK-Medium supplemented with 1 μg/ml TPCK trypsin was added and the infection was further incubated for 6 days. Afterwards the MDCK cells were analyzed for infection. The detection limit of the assay, determined by spiking with 100, 10 and 1 TCID₅₀ virus per flask, was approximately 1 TCID₅₀/ml.

Finding of lethal challenge dose of H1N1 wt in SCID mice: To define the lethal challenge dose 50 (LD₅₀) of wt H1N1 A/California/7/2009 in severe combined immunodeficient (SCID) mice, 4-5 week old female SCID mice (strain CB17/Icr-Prkdcscid/IcrCrl; Charles River, Sulzfeld, Germany) were used. They were challenged with 10-fold serial dilutions of the wt A/California/7/2009(H1N1) strain. The virus dose that kills 50% of the SCID mice (LD₅₀) was calculated by the software program Graph-Pad PRISM 5 (GraphPad Software Inc., La Jolla, Calif.).

H1N1 challenge and passive protection of SCID mice: For generation of sera for passive transfer studies, CD1 mice (Charles River) were immunized twice (d0, d21) with 10⁶ pfu recombinant MVA-H1-Ca, VV-L-H1-Ca, MVA wt or 3.75 μg of whole virus vaccines H1N1 A/California/07/2009, respectively. Serum pools were prepared on day 42 and analyzed via HI and ELISA. For passive protection experiments, 4-5 week old SCID mice were vaccinated intraperitoneally with 200 μl of the produced sera. One or two days afterwards, mice were challenged by intranasal instillation with 10⁵ TCID₅₀ per animal of the A/California/07/2009 (H1N1) wild-type strain and monitored for clinical parameters and survival for 30 days.

Hemagglutination Inhibition (HI) Assay: The HI titer of the sera was determined using chicken erythrocytes as described (9). Briefly, sera were treated with receptor destroying enzyme, inactivated at 56° C. and two-fold serially diluted. Sera were incubated with formalin inactivated A/California/07/2009 virus suspended to HA target titers of 3 followed by incubation with erythrocytes. Sera to be considered below detection limit (HI titer of 10) were assigned a nominal HI titer of 5.

Preparation of single cell Neuraminidase Inhibition (NI) Assay: Anti-neuraminidase serum antibodies were measured with a modified, miniaturized neuraminidase inhibition assay (Sandbulte et al., Influenza and Other Respiratory Viruses 3:233-240, 2009). Briefly, serially diluted inactivated H1N1 A/California/07/2009 wild-type virus preparation was mixed with an equal volume of fetuin (25 mg/ml) and incubated for 18 h at 37° C. After addition of periodate and incubation for 20 min, arsenite was added and the solution mixed until the brown color disappeared. Thiobarbituric acid was then added and samples boiled for 15 minutes. The pink reaction product was extracted by butanol and its absorbance determined at 550 nm using a reaction blank as reference. The half-maximal neuraminidase activity (EC₅₀) was determined using non-linear regression of the absorbance data (GraphPad PRISM, GraphPad Software Inc.). For the subsequent neuraminidase inhibition studies, the concentration of the virus was adjusted to an equivalent of half-maximal neuraminidase activity. Serially diluted sera were incubated with the appropriately diluted virus preparation for 1 hour and the neuraminidase activity determined as described above. The neuraminidase inhibition titer was defined as the reciprocal serum dilution at which neuraminidase activity was 50% inhibited suspensions from spleens or lungs of immunized and challenged mice: Single cell suspensions of splenocytes were obtained by grinding 5 spleens per group through a metal mesh into culture media (45% RPMI1640 (GIBCO®), 45% CLICKs Medium (Sigma), 10% FCS (GIBCO®), Penicillin-Streptomycin (GIBCO®), 2 mM L-glutamine (GIBCO®). Splenocytes were either used immediately or were frozen in Cryostor CS-10 (VWR, Bridgeport, N.J.). Single cell preparations were also obtained from the lungs of immunized mice before and after challenge with H1N1 swine flu virus. After euthanizing the mice, lungs were flushed by injecting 0.5-1 ml of PBS/Heparin (5 IE/ml) into the right ventricle of the heart. The lungs were then removed and immediately placed into culture media, cut into small pieces and digested for 25 min at RT by adding 5 mM MgCl2 (Merck, Whitehouse Station, N.J.), 150 Units/ml DNAse I (Roche, Pleasanton, Calif.) and 1 mg/ml collagenase XI (Sigma Inc.) to the culture media. After digestion, the remaining pieces were ground through a metal mesh into culture media, and the resulting cell suspension was filtered through a 70 mm nylon mesh (Becton Dickinson). Residual red blood cells were removed using RBC lysis buffer (Sigma Inc.) according to the manufacturer instructions, and the cells were used immediately for FACS-based IFN-γ analyses.

T-cell IFN-γ analysis: Peak response frequencies of vaccine-specific IFN-γ producing T-cells in Balb/c mice were determined by flow cytometric intracellular cytokine staining in spleens 7 days after the second immunization, or in lungs on the day before challenge (day 41) or 3 days after challenge (day 45). Approximately 2×106 cells were dispensed into 96-well round-bottom-plates (Costar, St. Louis, Mo.) and stimulated for approximately 14 h at 37° C. with vaccine antigens (H1N1/California/07/2009, H1N1/Brisbane/59/2007, H1N1/New Caledonia/20/99, H5N1/Vietnam/1203/2004) at 3 mg/ml hemagglutinin, or with peptide pools of 15-mers overlapping by 10 amino acids at 2 mg/ml per peptide. Two pools of 111 and 92 peptides were used, spanning the entire H1 or N1 proteins of A/California/07/2009, respectively. Cells incubated with medium alone served as a negative control. After 2 hours 10 mg/ml brefeldin A (Sigma Inc.) was added to inhibit secretion of cytokines and further incubated for 12 h. Cells were resuspended in 50 mM PBS/EDTA, and stained with LIVE/Dead Violet Kit (VIVID, Molecular Probes®, Carlsbad, Calif.) diluted 1:10000 in PBS for 15 min at room temperature. Cells were washed and incubated with rat anti-mouse CD4-APC antibody (BD Biosciences, 0.4 mg/mL), and CD8-APC H7 antibody (BD Biosciences, 1.5 mg/ml) for 10 min at room temperature. After washing and fixation with 1% paraformaldehyde (Merck), cells were permeabilized in PBS supplemented with 0.08% saponin (Sigma), and incubated with rat anti-mouse IFN-γ FITC antibody, (BD Biosciences, 0.5 mg/mL) and rat anti mouse CD3-PerCP (BD Biosciences, 1.5 mg/ml, San Jose, Calif.) for 30 min at room temperature. Finally, cells were washed and fixed with 1% paraformaldehyde. At least 100,000 viable cells were applied on a FACSCanto-2 (BD Biosciences), and data analysis was performed using the FlowJo software (Tree Star, Inc., Ashland, Oreg.). Percentages of IFN-γ producing T-cells were calculated after gating on VIVID negative, CD3 positive, CD4 or CD8 positive lymphocytes.

Microneutralization assay: The microneutralization assay was done as described previously (Kistner et al., PLoS One 5:e9349, 2010). Briefly, sera was diluted and mixed with the A/California/7/09 virus strain at a concentration of 4.5 log TCID50/ml. The mixture was incubated for six days on MDCK monolayer before cells were inspected for cytopathic effects. The neutralizing antibody titer was defined as described (Kistner et al., PLoS One 5:e9349, 2010).

Statistical analysis: For statistical comparison of lung titers the Tukey test was used. The data of T cell responses were statistically analyzed using the two-way ANOVA. Survival differences between animal groups were analyzed using the Kaplan Meyer log rank test of GraphPad PRISM software. All differences were considered significant at P values <0.05.

Results

1. Construction and Characterization of the MVA Viruses

The HA and NA genes of the influenza strain A/California/07/2009(H1N1) were placed downstream of a strong vaccinia early/late promoter (22) and the resulting plasmids were used to construct the viruses MVA-H1-CA and MVA-N1-CA by in vivo recombination techniques using transient marker genes. Further, replicating control viruses based on the vaccinia Lister strain, rVVL-H1-CA and rVVL-N1-CA, respectively, were constructed (see Table 2 and Methods). The H1 and N1 genes were synthetic genes optimized for expression in vaccinia virus, lacking internal transcription stop signals. The virus constructs were characterized by PCR for the absence of wild-type virus and for the presence of the HA and NA gene inserts.

TABLE 2
Viruses and vaccines constructs and controls used in the study.

| | | | Titer^{b} |
|---|---|---|---|
| Vaccine | inserted flu gene | plasmid | (pfu/ml) |
| MVA-H1-CA | Hemagglutinin | pHA-mH5-H1-Ca^{a} | 4.0 × 10⁹ |
| MVA-N1-CA | Neuraminidase | pHA-mH5-NA-Ca | 1.6 × 10⁹ |
| rVVL-H1-CA | Hemagglutinin | pER-mH5-H1-Ca | 4.3 × 10⁹ |
| rVVL-N1-CA | Neuraminidase | pER-mH5-NA-Ca | 7.4 × 10⁹ |
| VV-WT (Lister) | empty vector | n.a. | 1.7 × 10¹¹ |
| MVA-wt | empty vector | n.a. | 5.0 × 10⁹ |
| Inact. whvv^{f} | n.a. | n.a. | n.a. |

^{a}influenza genes in VV constructs are controlled by the vaccinia mH5 promoter; HA and NA genes derived from influenza CA/07 strain;
^{b}titers of sucrose-purified virus preparations (see Methods);
^{c}MVA parental strain NIH-LVD clone 1 . . . ;
^{f}inactivated whole virus vaccine;
n.a. , not applicable.

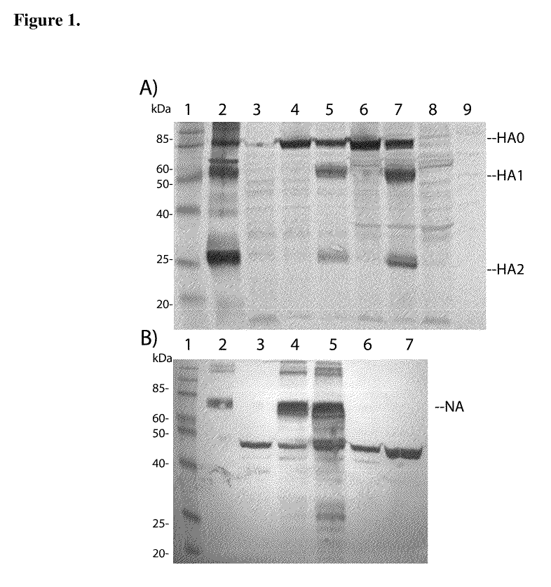

Next, expression of the influenza genes in avian and mouse cells was analyzed. To measure parameters such as temporal order and the level of gene expression and processing of the protective antigen influenza immune responses in vaccinia-based live vaccines, western blot analyses with lysates of infected cells were performed. In infected target cells of the respiratory tract, the influenza hemagglutinin precursor (H0) is cleaved by intracellular proteases into the heterodimeric receptor molecule (H1 and H2) resulting in full infectivity of influenza virus. In order to assess HA antigen levels and processing, expression experiments in MVA-permissive avian cells and in nonpermissive murine muscle cells were performed. Total cell lysates were analyzed by PAGE and Western blotting using anti-hemagglutinin sera. As shown in FIG. 1A, the viruses with HA gene inserts induced high level expression of HA in avian DF-1 cells. The large band at around 80 kDa represents the H0 hemagglutinin-precursor that was not cleaved in cells lacking the proper proteases such as the chicken cells used to propagate MVA (lanes 4 and 6). To further characterize the H0 precursor, the cell lysates were treated with trypsin (see Methods). In both, the VVL-H1-CA and the MVA-H1-CA-infected cells, two novel bands at approximately 64 and 26 kDa appeared representing the HA1 and HA2 subunits (lanes 5 and 7). The polypeptide pattern of the inactivated whole virus vaccine is shown in lane 2. The HA-specific bands in this control co-migrate with the ones expressed by the viral vectors. In the negative controls these bands are absent (lanes 3, 8, 9). A similar analysis was performed in mouse muscle cells, infected with the HA containing vectors and controls. Again, in murine cells only the HA0 band could be seen, which was then cleaved into the two subunits upon trypsin treatment. Thus, the same expression pattern was obtained in cells permissive for MVA and in nonpermissive murine cells that presumably reflect the situation in mice vaccinated by the intramuscular route.

In order to confirm the expression pattern of the neuraminidase constructs, lysates of infected DF-1 cells, together with controls were subjected to western blotting and probed with an antibody raised against a peptide present in the neuraminidases of different subtypes including N1 and N5 (see Methods). The recombinant vectors induced a broad novel band in the 75 kDa range, representing the highly glycosylated neuraminidase of the CA/07 strain (FIG. 1B, lanes 4 and 5), that was also detectable in the inactivated vaccine preparation (lane 2), but was absent in the negative controls including non-infected and wild-type virus infected cells (lanes 3, 6, 7). The prominent band in the 47 kDa size range (lanes 3-7) is a protient present in DF-1 cells non-specifically cross-reacting with the primary antibody.

### 2. Protection Studies in Immune Competent Mice

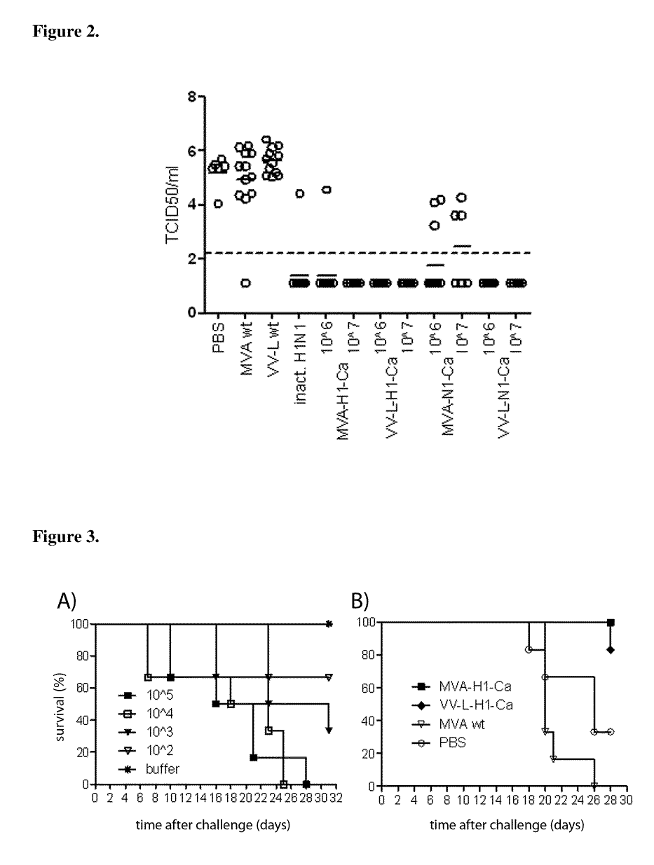

The ability of the vaccine to protect BALB/c mice from infection was then measured. Upon challenge with H1N1 swine flu virus, this mouse strain develops signs of disease including pneumonia, however, mice recover after two weeks (11). In this model, protection from pneumonia, indicated by absence of virus in the lungs, was used as the read-out for efficacy. In the protection experiment the hemagglutinin- and neuraminidase-expressing viral constructs were compared to a formalin-inactivated whole virus (whv) vaccine. The live vaccines were given by intramuscular injection of 1×10⁶ and 1×10⁷ pfu per animal, the dose of the inactivated vaccine was 3.75 ug (Table 3). The groups were boosted after 21 days and challenge of all groups was carried out at day 42. Negative controls included mice immunized with the empty vectors (MVA-wt and VV-L) and with phosphate buffered saline (PBS). The challenge virus was given intranasally at a dose of 1×10⁵ TCID50 per animal. Three days after challenge the lungs were removed and titers were determined. The results of the lung titrations are shown in FIG. 2 and in Table 3. In the groups immunized with the hemagglutinin constructs, MVA-H1-CA or rVV-L-H1-CA, protection from pneumonia was nearly complete, approximately 92-100% protection was achieved, depending on dose and construct (Table 3, groups 1-4). Hemagglutinin-inhibition (HI) titers of the mouse sera were in the range 453-905. Neutralization titers were also high, i.e., in the range of 761-1076.

TABLE 3
Protection of mice from pneumonia and serology results after two vaccinations.

| | | | protection | HI- | | NI- | ELISA |
|---|---|---|---|---|---|---|---|
| | | dose | n/ntotal | Titer⁽¹⁾ | μNT⁽³⁾ | Titer⁽⁴⁾ | total |
| Gr. | Virus | [pfu/mouse] | (%) | [GMT⁽²⁾] | [GMT⁽²⁾] | [GMT⁽²⁾] | IgG |
| 1 | vMVA-H1-CA | 10\hat{ }6³ | 11/12⁽⁵⁾ | 453 | 905 | n.d. | 1022 |
| | | | (92) | | | | |
| 2 | vMVA-H1-CA | 10\hat{ }7 | 12/12 | 905 | 1076 | n.d. | 1310 |
| | | | (100) | | | | |
| 3 | rVVL-H1-CA | 10\hat{ }6 | 12/12 | 453 | 761 | n.d. | 1039 |
| | | | (100) | | | | |
| 4 | rVVL-H1-CA | 10\hat{ }7 | 12/12 | 640 | 1076 | n.d. | 1218 |
| | | | (100) | | | | |
| 5 | vMVA-N1-CA | 10\hat{ }6 | 9/12 (75) | <dl⁽⁹⁾ | n.d. | 128 | <0.04 |
| 6 | vMVA-N1-CA | 10\hat{ }7 | 4/12 (83) | <dl | n.d. | 1024 | <0.04 |
| 7 | rVVL-N1-CA | 10\hat{ }6 | 12/12 | <dl | n.d. | 1024 | <0.04 |
| | | | (100) | | | | |
| 8 | rVVL-N1-CA | 10\hat{ }7 | 12/12 | <dl | n.d. | 2048 | <0.04 |
| | | | (100) | | | | |
| 9 | In. Whvv⁴ | 3.75 | 11/12 (92) | 640 | 538 | 256 | 477 |
| 10 | MVA-wt⁵ | 10\hat{ }7 | 1/12 (8.3) | <dl | <dl | <2 | <0.04 |
| 11 | VVL-wt⁶ | 10\hat{ }7 | 0/12 (0) | <dl | n.d. | <2 | <0.04 |
| 12 | PBS | — | 0/12 (0) | <dl | <dl | <2 | <0.04 |

⁽¹⁾hemagglutinin-inhibition titer determined with chicken erythrocytes;
⁽²⁾geometric mean titer;
⁽³⁾immunizations with a dose of 10\hat{ }6 were repeated once.
⁴inactivated whole virus vaccine, route sc; (monovalent bulk lot17);
⁵wild-type MVA (NIH74 LVD clone 6);
⁶wild-type vaccinia Lister strain.

With the neuraminidase construct, MVA-N1-CA, partial protection was achieved. Even in the unprotected mice the lung virus titers in were rather low, indicating some degree of protection in these animals also (FIG. 2 and Table 3, groups 5-6). With the replicating vaccinia construct, rVVL-N1-Ca, full protection was seen at both doses (Table 3, groups 7-8). Neuraminidase-inhibiting antibodies were high after immunization with the viral constructs expressing NA, and absent in the HA constructs used as controls. As expected, the HI titers were below the detection limit. Mice immunized with the inactivated vaccine were almost fully protected with HI titers of 640 and NI titers of 256 (Table 3, group 9). Mice injected with the negative controls, empty MVA and VVL vectors and PBS, were not protected and sera were negative for HI antibodies (groups 10-12). The number of protected mice in the groups immunized either with inactivated vaccine, or the live vaccines was significantly higher as compared to the controls (P<0.001).

In order to further compare the vaccines, single dose immunizations were also carried out followed by challenge at day 42. As shown in Table 4, only the live vaccines protected mice from virus replication in the lungs. With the vaccinia-based vaccines 100% and 83% protection was achieved with the replicating vaccinia vectors and with the MVA-based vaccines, respectively, while the inactivated vaccine did not prevent replication in the lungs. In case of the NA viruses, protection was less efficient, 33% and 67% protection was obtained with the replicating and the nonreplicating vectors, respectively (Table 4).

TABLE 4
Protection (lung titer) and serology results
of mice after single dose vaccinations.

| | | dose | protection | HI-Titer¹ | ELISA³ |
|---|---|---|---|---|---|
| Gr. | Virus | [pfu/mouse] | [n/ntotal (%)] | [GMT²] | [GMT] |
| 1 | vMVA-H1-CA | 10\hat{ }6 | 10/12 (83) | 120 | 218 |
| 2 | vMVA-N1-CA | 10\hat{ }6 | 8/12 (67) | 5 | <DL⁴ |
| 3 | rVVL-H1-CA | 10\hat{ }6 | 6/6 (100) | 160 | 203 |
| 4 | rVVL-N1-CA | 10\hat{ }6 | 2/6 (33%) | <DL | <DL |
| 5 | In. Whvv⁵ | 3.75 | 0/6 (0%) | 80 | <DL |
| 6 | PBS | — | 0/12 (0) | 5 | <DL |

¹hemagglutinin-inhibition titer determined with chicken erythrocytes;
²geometric mean titer;
³total IgG, recombinant baculovirus-derived H1 used for coating;
⁴<DL, below detection limit;
⁵inactivated whole virus vaccine, monovalent bulk lot 17.

3. Induction Kinetics of Antibody Responses

Since induction of rapid immunity is crucial for pandemic vaccines, the kinetics of antibody induction against the major protective antigen, the hemagglutinin, was determined after single dose vaccinations. For this purpose, mice were immunized with increasing doses (range 10⁶ to 10⁸ pfu/animal) of the H1 containing vectors and with appropriate controls. Sera were taken each week over a period of 70 days, pooled by groups and analyzed using the HI- and μINT-test (Table 5). With MVA-H1-Ca, the first HI antibodies were detectable at day 7 (Table 5, group 3), at that time point the neutralizing antibodies were below the detection limit. Apparently independent of dose, both, neutralizing and HI antibodies were found after day 21. In the examined dose range, titers increased over time, however no clear dose dependence was seen. The titers induced by the replicating control, rVVL-H1-Ca, were clearly dose dependent. NT and HI titers were detectable starting at day 7 in the 10⁸ pfu dosage group (Table 5, group 6). Titers increased with time and remained high over the observation period. With the controls, MVA wt and PBS, the titers remained below the detection limit.

TABLE 5
Geometric mean neutralization (NT) and hemagglutination inhibitions (HI) titers of
mouse sera after single dose immunizations with the live vectors.

| | | | Day 7⁽¹⁾ | Day 14 | Day 21 | Day 28 | Day 35 | Day 42 | Day 70 |
|---|---|---|---|---|---|---|---|---|---|
| Gr | Vaccine | Dose | [NT/HI] | [NT/HI] | [NT/HI] | [NT/HI] | [NT/HI] | [NT/HI] | [NT/HI] |
| 1 | MVA-H1- | 10⁶ | <dl/<dl | <dl/14 | 80/40 | 57/67 | 320/80 | 160/113 | 453/226 |
| | Ca | | | | | | | | |
| 2 | MVA-H1- | 10⁷ | <dl/<dl | <dl/10 | 40/40 | 28/57 | 160/80 | 160/95 | 320/160 |
| | Ca | | | | | | | | |
| 3 | MVA-H1- | 10⁸ | <dl/10 | 20/20 | 80/80 | 113/113 | 226/160 | 160/160 | 640/320 |
| | Ca | | | | | | | | |
| 4 | rVV-L-H1- | 10⁶ | <dl/<dl | <dl/<dl | 57/28 | 40/57 | 80/113 | 57/80 | 453/160 |
| | Ca | | | | | | | | |
| 5 | rVV-L-H1- | 10⁷ | <dl/40 | 40/57 | 20/80 | 160/113 | 160/226 | 640/226 | 1280/226 |
| | Ca | | | | | | | | |
| 6 | rVV-L-H1- | 10⁸ | 28/57 | 113/80 | 28/160 | 226/160 | 320/226 | 320/320 | 1810/320 |
| | Ca | | | | | | | | |
| 7 | MVA-wt⁽²⁾ | 10⁶ | <dl/<dl | <dl/<dl | <dl/<dl | <dl/<dl | <dl/<dl | <dl/<dl | <dl/<dl |
| 8 | PBS | — | <dl/<dl | <dl/<dl | <dl/<dl | <dl/<dl | <dl/<dl | <dl/<dl | <dl/<dl |

⁽¹⁾day 0 titers were all below the detection limit (dl);
⁽²⁾wild-type MVA (NIH74 LVD clone 6); detection limits of the HI and the NT assay were <10.

4. Passive Protection Studies in Severe Combined Immune-Deficient (SCID) Mice

Animal models should reflect the pathology induced in humans. In the case of the new H1N1 influenza, the model should protect from typical disease symptoms, such as malaise, pneumonia, but also from death. Since immune competent Balb/c mice and even more susceptible CD1 mice recover from wt H1N1 challenge, SCID mice were used in the following experiments. For the passive protection experiments, first, antisera were generated. Immune competent mice (Balb/c) were immunized twice with the MVA-H1-CA and VVL-H1-CA vectors and sera were harvested at day 42. The HA-specific sera used for transfer had HI titers of 640 while the titer of the control serum (MVA-wt vaccinated mice) was below the detection limit.

Next the virulence of the new H1N1 strain in SCID mice was analyzed. After intranasal challenge with the A/California/07/2009(H1N1) strain, the SCID mice died in a dose-dependent manner from pneumonia. Doses of 10⁴ TCID50 per animal killed all mice within a four week period (FIG. 3A). In parallel, SCID mice were treated with the antisera. All mice treated with the MVA-H1 derived sera were fully protected from death and disease symptoms while the protection rate of animals treated with the VVL-H1-CA-derived sera was 83%. The control animals, however, got sick and died in the third and fourth week after infection (FIG. 3B). In some of the animals protected by the MVA-H1-CA-induced sera, no virus was found in lungs at the end of the experiment at day 30. In others, despite lack of clinical symptoms, virus was present in the lungs. Thus, passively transferred antibodies, induced by the MVA-H1-CA live vaccine and the replicating controls, protect mice from lethal challenge with the wild-type CA/07 virus confirming the important role of anti-H1 antibodies in protection from H1N1 influenza. Full passive protection of SCID mice from lethal challenge by antisera generated in mice and guinea pigs with the inactivated H1N1 whole virus vaccine used also in this study was seen previously (8).

5. Hemagglutinin-Specific and Cross-Reactive T Cell Responses

In order to investigate T cell responses induced by the different vaccines, mice were immunized twice (days 0 and 21) with the different vaccines. Splenocytes were prepared on day 28 and stimulated in-vitro with whole virus antigens or HA- or NA-specific peptide pools of overlapping 15-mers covering the entire HA or NA sequence of the CA/07 H1N1 strain. The whole virus antigens used for stimulation included the monovalent vaccine bulks (MVBs) of inactivated influenza preparations of the H1N1 strains CA/07 (H1N1/CA), A/Brisbane/59/2007 (H1N1/BR), A/North Carolina (H1N1/NC) and the H5N1 strain A/Vietnam/1203/2004 (H5N1/VN). The number of INF-γ producing T cells was then determined by FACS-based intracellular cytokine assays. Differentiation between CD4 and CD8 T cells was achieved by staining of T cell specific markers (see methods).

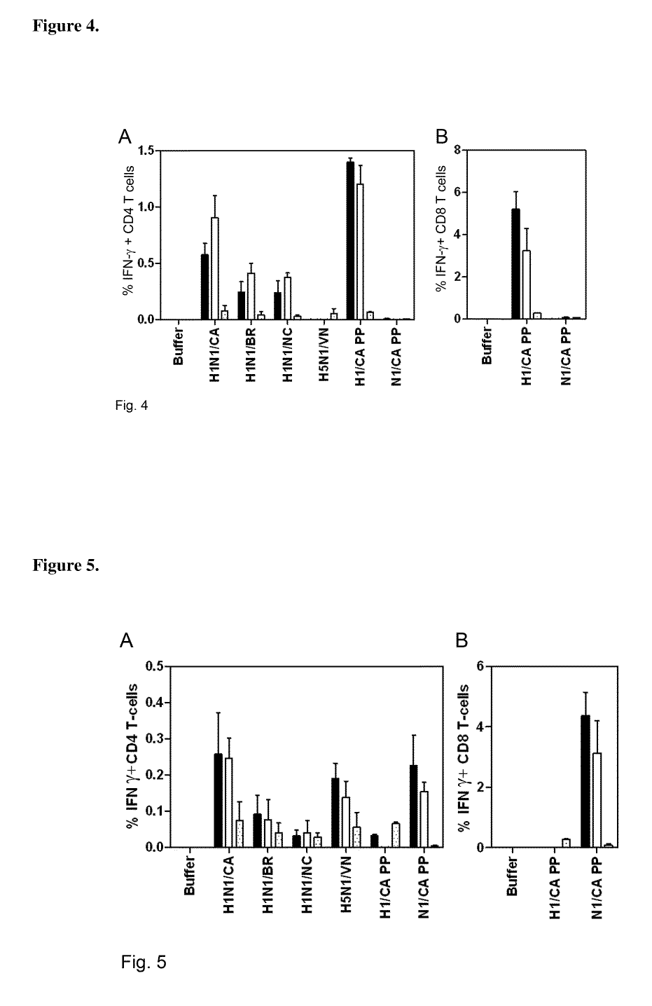

The results obtained with hemagglutinin-containing constructs MVA-H1-CA and VVL-H1-CA and with the inactivated vaccine are shown in FIG. 4. Specific induction of 0.5-0.8% of CD4 T cells was seen after homologous stimulation with H1N1/CA antigen of splenocytes of mice vaccinated with the MVA and VVL-based vaccines. Significant levels of cross-induction were still achieved with the H1 Brisbane and North Carolina antigens. The highest levels of CD4 T cell stimulation were seen, however, with the HA-CA/07-specific peptide pools. With both the MVA and the VVL-based HA construct 1.3-1.4% of the CD4 T-cells produced IFN-γ secretion in mice immunized with both the MVA and the VVL-based HA constructs. The levels achieved with the inactivated virus were several-fold lower (FIG. 4A).

Next, CD8 T cells induced by the HA constructs were analyzed. The H1/CA peptide pool stimulated surprisingly high amounts of CTLs, around 3-5% of total CD8 T cells were specific for H1, while the N1-specific peptide pool used as a negative control did not induce significant levels of CD8 T cells (FIG. 4B). The MVA construct induced higher levels of CD8 T cells compared to the Lister construct. The inactivated vaccine induced low but measurable amounts of CTLs. The splenocytes of mice vaccinated with the empty vectors did not react with the HA-specific antigens and peptides. An HA2-specific epitope conserved in the HAs of influenza subtypes 1, 5 and 9 (HA534-42) was also used in this study. Specific induction of CD8 T cells in the range of 2-3% were seen with this peptide. Since the total peptide response of the pool was about 3-fold higher, more HA peptides should contribute to the strong CD8 T cell response.

6. Neuraminidase-Specific T Cell Responses

Further, neuraminidase as target of T cells was analyzed. Mice were immunized as described above with neuraminidase-containing constructs MVA-N1-CA and VVL-N1-CA and with the controls. The results obtained are shown in FIG. 5. Using the homologous stimulant, H1N1/CA antigen, induction of about 0.25% of CD4 T cells by the live vectors was achieved. With the Brisbane H1N1 and with the H5N1 antigens, good levels of cross reactivity were seen, while the North Carolina antigen was a poor stimulant (FIG. 5A). A peptide pool of 15 mers spanning the whole N1 sequence induced amounts of CD4 T cells comparable to the homologous proteinaceous antigen. Again the inactivated vaccine induced lower amounts of T cells. Interestingly, a very strong CD8 T cell response against N1 was also found in mice vaccinated with the N1-containing live vaccines (FIG. 5B). Three to four percent of total CD8-T cells were N1-specific, when the N1/CA04 peptide pool was used for stimulation, while the H1 peptide pool was negative. As seen before, induction of T cells by the inactivated vaccine was far lower.

7. Hemagglutinin-Specific T Cell Responses in the Lung Before and 3 Days After Intranasal Challenge with H1N1 Swine Flu Virus

To analyze whether the live vaccines, besides inducing high levels of influenza-specific CD4 and CD8 T-cells in the spleen shortly after booster immunization, also give rise to significant levels of HA-specific T-cells at the site of influenza infection, influenza-specific interferon γ-secreting T cells were also quantified in the lungs. Mice (5 per group) were immunized twice (at days 0 and 21) with the hemagglutinin constructs (MVA-H1-Ca and rVVL-H1-Ca), and with the MVA wt control (10⁶ pfu per animal). Subsequent challenge with H1N1 wt virus was carried out at day 42. The pre- and post challenge lung T cells were analyzed. For comparison reasons, T-cell responses were also measured at the same time points in the spleens, a site not directly involved in viral infection.

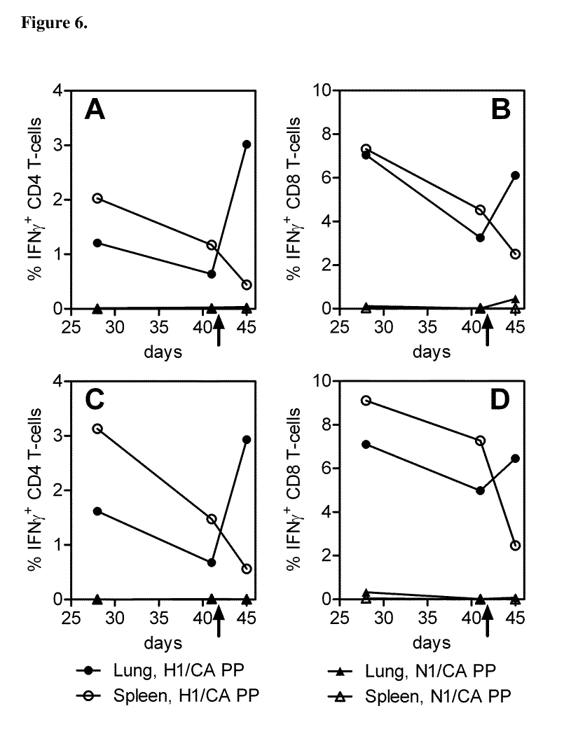

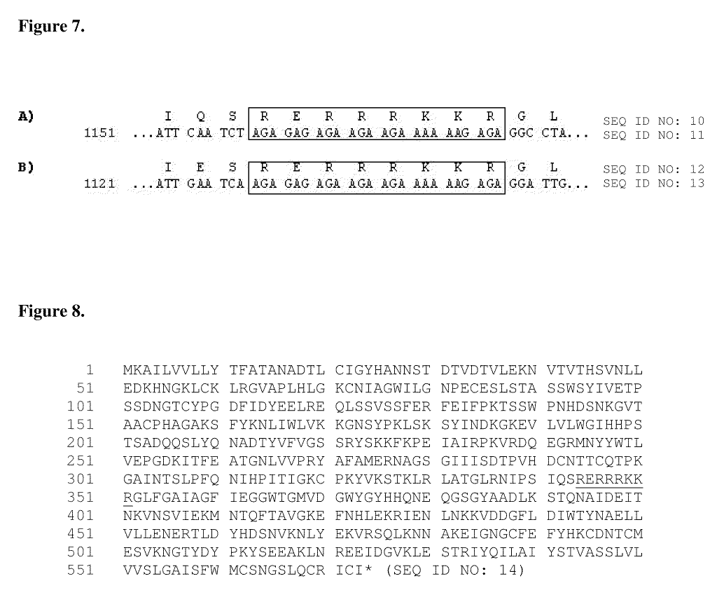

Seven days after the second immunization, and one day before and three days after the challenge (at days 28, 41 and 44), lungs and spleens were isolated and cells processed for analysis. Mice were given an anesthesia, lungs were flushed in-vivo with a heparin-containing medium to remove blood cells (that might have an impact on lung-specific T cell counts) and lungs and spleens were collected. Single cell suspensions were prepared from 5 pooled lungs or spleens of immunized mice. Pulmonary and spleen cells were stimulated with the HA- and the control peptide pools (H1/CA PP and N1/CA PP, respectively) and subjected to FACS analysis. High frequencies of influenza-specific CD4 and CD8 cells were induced by vaccination with the MVA-based HA vaccine, not only in the spleens but also in the lungs, although the frequencies were slightly lower in the lungs (FIG. 6). Seven days after the booster immunization, approximately 1% of the lung CD4 T cells (FIG. 6A) and 7% of the lung CD8 T-cells (FIG. 6B) were HA specific. Two weeks later, levels of T-cells had consistently dropped by 40-50% in both lungs and spleens. Interestingly, however, three days after the challenge, a five-fold increase in IFN-γ expressing HA-specific CD4 T cells was found only in the lungs. In contrast, depletion of the spleens from influenza specific CD4 T-cells was observed at that time point, within three days after infection CD4 frequencies have dropped by 60% (FIG. 6A).

A similar result was seen for the CD8 T cells. Before challenge, about 3% of the CD8 pulmonary T cells were HA-specific, and a two-fold increase was detected after challenge with H1N1 wt virus at day 45, whereas the CD8 T-cell frequency in the spleens decreased by 50%. The NA peptide pool (N1/CA PP), used as a control in this experiment, induced only background levels of T cells, demonstrating that little or no T-cells induced by the infection with the swine virus were present in the lungs three days after challenge. Therefore, the observed increase of influenza specific T-cells in the lungs upon challenge presumably reflects influx of the HA-specific effector T-cells from other sites into the lung.

Consistent results were obtained in mice immunized with rVV-H1-Ca live vaccine. Also in this group, a pronounced rise in influenza-specific CD4- (FIG. 6C) and CD8 T-cells (FIG. 6D) was observed after challenge with swine influenza virus. As expected, immunization with the MVA wt control did not induce any influenza-specific T-cells in the lungs before and after challenge. This clearly demonstrates that functionally competent effector T cells were induced by the vaccine which were capable of homing into virus-infected tissue such as the lungs.

Discussion

In the above study, experimental vaccinia-based pandemic H1N1 influenza live vaccines were compared to an inactivated whole virus vaccine. When given in two doses, the hemagglutinin containing vaccines were fully protective in active immunizations and protected animals had high HI antibody titers. The neuraminidase containing MVA viruses were partially protective.

After single dose vaccinations only the HA-containing live vaccines induced protection from virus in the lungs, presumably due to the much higher T cell responses induced by these vaccines. Earlier work indicates that CD8 T cells contribute to viral clearance of influenza in the lungs of mice and thus contribute to protective immunity (23) and more recent work in chickens suggests, that pulmonary cellular immunity is very important in protecting naive natural hosts against lethal H5N1 influenza viruses (16). T-cell responses to natural influenza infections are mainly directed against common epitopes on the nucleoprotein, PB2 and M1 but also against the highly variable haemagglutinin and the neuraminidase (21).

The MVA-based live vaccines induced surprisingly high amounts of CD8 T cells, both the HA and the NA constructs gave rise to 4-5% of total antigen-specific CD8 T cells in splenocytes 7 days after the second immunization, which may explain the good performance after single dose vaccinations. Further, the HA antigen-specific INF-y producing CD4 T cells were higher in the live vaccines. Considerable levels of CD4 T cells cross-reacting with related H1 strains were found while cross-reaction with the H5 subtype was not seen. CD4 T cells induced by the recombinant N1 vectors also cross-reacted with the neuraminidase for seasonal H1N1 strains, and also against the N1 of the avian VN1203 H5N1 strain.

Furthermore, high levels of HA-specific CD4 and CD8 T-cells were detected in the lungs of mice immunized with the MVA-HA vaccine construct at the time of challenge with wild type H1N1/CA virus, with a further accumulation of effector T-cells observed in the lungs three days after the challenge. Accumulation of specific effector T-cells in the lungs of infected mice has been shown to depend on differential expression of homing receptors such as CD44 or CD62L (Cerwenka et al., J Immunol 163:5535-43, 1999), which target the cells from the lymphoid tissue to non-lymphoid organs. However, upon pulmonary infection with influenza virus, IFN-γ secreting CD8 T-cells are detected only after 5 days in the lungs (Lawrence et al., J. Immunol. 174:5332-5340, 2005) whereas, after immunization with MVA-H1, they are present already in high frequencies before infection, and, upon infection, enter the lungs much more rapidly by relocation from other sites such as the spleen. These influenza-specific effector T-cells present at the site of infection have the potential to contribute to protection in various ways, for instance, by inhibition of viral replication, by secretion of cytokines, by direct killing of virus-infected host cells, or by support of influenza specific B-cells. Interestingly, in Balb/c mice, the primary pulmonary CD8 T cell response against influenza A/Japan/305/57(H2N2) appeared directed against three epitopes in the HA and one in the NP (Lawrence et al., J. Immunol. 174:5332-5340, 2005), suggesting that CD8 T cell responses are strongly dependent on the infecting virus strain and the host genetic background. Moreover, lung-resident proliferation contributes significantly to the magnitude of the antigen-specific CD8 T cell response following influenza virus infection (McGill et al., J. Immunol. 183:4177-4181, 2009).

A further advantage of using a poxviral (e.g. MVA) vector as pandemic influenza vaccine is its genetic stability including the expressed foreign genes. Passage of influenza primary isolates in eggs usually results in adaptive mutations in the HA gene. When non egg-adapted human influenza virus, i.e., either the natural virus present in a clinical specimen or an isolate propagated exclusively in tissue culture cells, is first passaged in the allantoic cavity of embryonated hens' eggs, variants which have amino acid substitutions around the receptor binding site are selected (19). Therefore, egg adaption can result in altered antigenicity resulting in less protection (21). Similarly, influenza virus grown in cell lines selects for specific variants present in the seed viruses, that may not be the preferred ones present in the human isolate. Therefore, to preserve the original genotype of viruses used as reference strains, a complex procedure is recommended, involving cloning in chicken eggs of the candidate virus at a very early passage, selection and analysis with appropriate antibodies and selection of the isolate whose hemagglutinin molecule most closely resembles the clinical isolate (3). All these complications are overcome if a stable DNA virus, such as MVA, is used as a vector for the influenza genes. This vector is independent of influenza virus-specific selection mechanisms and thus, the originally inserted sequences do not change resulting in preservation of originally inserted the HA or NA genes.

MVA-based pandemic vaccines may also have advantages as compared to cold-adapted live influenza vaccines. In a recent evaluation of live attenuated cold-adapted H5N1 influenza virus vaccines in healthy adults, HI and neutralizing antibody responses were found to be minimal (6). Several reasons were identified for this failure, the attenuating mutations specified by the A/Ann Arbor/6/60 (H2N2) cold-adapted virus loci had the greatest influence, followed by the deletion of the H5 HA multi-basic cleavage site (MBS), and the constellation effects of the internal backbone genes acting in concert with the H5N1 glycoproteins (17). In poxviral vectors, such influenza virus-specific reasons do not play a role. Injection of defined amounts of a stable vector results in reliable delivery and the use of strong vaccinia promoters and optimized foreign genes result in high-level expression and good induction of B and T cell responses. In addition, MVA tolerates pre-existing anti-vaccinia immunity and can be used as an immunizing agent under conditions of pre-existing immunity to the vector and thereby may allow repeated use (13).

Moreover, after passive transfer of sera from MVA-H1 immunized mice, full protection of SCID was achieved while the control mice, challenged with a dose of 1×10⁵ TCID50 of wt CA/07 virus died within three weeks from pneumonia and titers of 5-6 log 10 TCID50 were found in the lungs. Interestingly, despite lack of clinical symptoms in the protected SCID mice, in the majority of lungs virus was found at the end of the 4 week monitoring period. This indicates that a single prophylactic dose of antiserum given prior to challenge was not sufficient for virus clearance. However, as shown previously, infection of SCID mice with A/PR8/34(H1N1) influenza virus followed by passive transfer two doses of an anti-hemagglutinin antibody cocktail at days 2 and 6 led to clearance and full recovery from infection (15). Thus, protection, including clearance of virus, may be achieved by a higher dose and by repeated dosing of the antiserum. Further, in the case of H1N1 CA/07 virus, full protection of SCID mice was obtained after passive transfer of sera generated with the inactivated whole virus vaccine in mice and guinea pigs (8).

The mouse data described above suggests that modified vaccinia Ankara-based recombinant viruses, besides extraordinary safety, have some further advantages as pandemic influenza vaccines, including excellent induction of antibodies and T cells, genetic stability of the hemagglutinin due to independence from influenza-specific genetic alterations and efficacy after single dose vaccination. The present study shows that MVA is a good alternative as pandemic influenza vaccine against the novel H1N1 subtype, in accordance with previous studies demonstrating that corresponding MVA recombinants also protect against avian H5N1 strains (Kreijtz et al., J Infect. Dis. 195:1598-1606, 2007; Kreijtz et al., J Infect Dis 199:405-13, 2009). The H1N1 pandemic strain currently causes mild disease presumably due to partial immunity mainly in the adult population. If, similar to the 1918 pandemic, more virulent mutants would occur in subsequent waves of infection, more broadly protective vaccines are desirable.

Recombinant vaccinia viruses, antigenic compositions or vaccines of the present invention are administered to human subjects using techniques known in the art.

### Example 2

### Introduction of a Modified HA1/HA2 Cleavage Site Into the Hemagglutinin Gene and Characterization of the Virus (H1 Swine Flu and H2 of Singapore Strain)

For full receptor function of the influenza virus hemagglutinin (HA), cleavage of the HA precursor into the subunits HA1 and HA2 is required. This usually occurs in the airway epithelia of the respiratory tract. The HA receptor acquires its final conformation and the influenza virus gains its infectivity. Vaccinia-based live vaccines are usually administered by the subcutaneous or intramuscular routes. In the cell types present in this environment (subcutaneous tissue, muscle) cleavage of the HA0 precursor usually does not occur. On the other hand, the H5 hemagglutinins of the avian H5N viruses (for example the A/Vietnam/1203/2004 strain) contain a polybasic cleavage site that is readily cleaved in cell types that do not contain the proteases normally required for cleavage. Thus, introduction of a polybasic cleavage site into a HA, that normally does not contain such a site, results in fully functional receptors having their natural conformation when expressed in non-airway tissues.

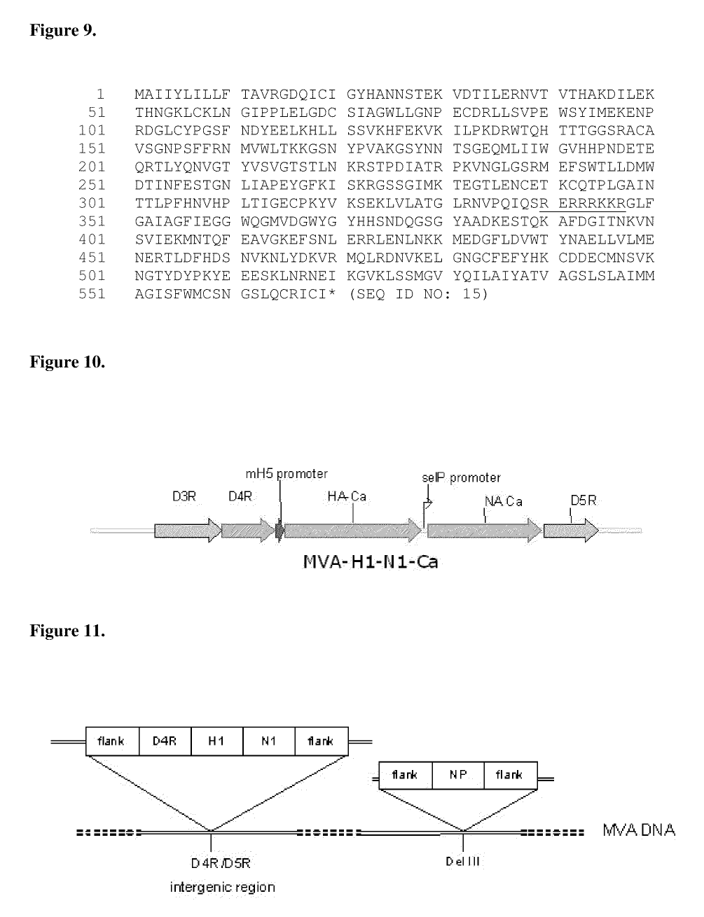

In order to express the completely functional HA, the cleavage site of the HA is modified (see FIGS. 7-9). The modified HA contains a more efficient cleavage site recognized by furin-like ubiquitous proteases. After intramuscular injection of the live vaccine, the HA0 induced by the vaccine is completely cleaved and presents the novel conformational epitopes that widen the antibody response. Surprisingly, using MVA as a vector, a higher expression level is achieved with the modified HA, and in turn, a better overall immune response. In vaccinia-based live vaccines, presence or absence of the polybasic cleavage site in the influenza HA molecule does not affect infectivity or safety of the live vaccine.

This type of modification can be done with all HA subtypes that normally do not possess polybasic cleavage sites. Influenza HAs of subtypes H5 and H7 are known to naturally possess such sites and they are not necessarily associated with increased virulence (30). All other HAs usually do not possess such cleavage sites (but may acquire them naturally).

Materials and Methods

Cells and Viruses: Cell lines. The DF-1 (CCL-12203) cell line is obtained from the American Type Culture Collection. The cell line is cultivated in DMEM (Biochrom AG, Berlin, Germany) containing 5% fetal calf serum (FCS). Chicken embryo cells (CEC) are cultivated in M199 (GIBCO®, Inc.) containing 5% fetal calf serum (FCS). Madin-Darby canine kidney (MDCK) cells are maintained serum free in ULTRA-MDCK medium (Bio Whittaker®).

Influenza strains. The influenza virus A/California/07/2009 (H1N1; CDC #2009712112) was kindly provided by the Centers for Disease Control and Prevention (CDC, Atlanta, USA). The influenza virus A/Singapore/1/1957(H2N2) is obtained from the Centers for Disease Control and Prevention (CDC, Atlanta, USA) or from another collection of microorganisms.

Cloning of the wild-type and modified hemagglutinin genes. The hemagglutinin sequences of A/California/04/2009 (Genbank accession #FJ966082) and of A/Singapore/1/1957 (Genbank accession #CY034044) are chemically synthesized (Geneart, Regensburg, Germany). The synthetic gene includes the strong early/late vaccinia virus promoter mH5 upstream of the coding region and a vaccinia virus specific stop-signal downstream of the coding region. The gene may be codon-optimized for high expression. For the MVA insertion, the HA gene cassette is cloned into plasmid pHA-vA (24) resulting in pHA-mH5-H1-Ca. The insertion plasmid directs the gene cassette into the MVA HA-locus.

The modified cleavage sites are introduced by PCR mutation using the wild-type sequence as a template. For this purpose PCR (KOD Hot Start PCR Kit, Novagen, EMD Chemicals Inc., Gibbstown, N.J.) is performed to mutate the original cleavage site using the oligo pairs o.HA-Ca5 (GGCACCTATA AATTGGGCTC AAGG) (SEQ ID NO: 2) plus o.HA-Ca8 (CTTTTTTCTT CTTCTCTCT CTAGATTGAA TAGACGGGA) (SEQ ID NO: 3) and o.HA-Ca6 (AATTTCACTA AAGCTGCGG CCG) (SEQ ID NO: 4) plus o.HA-Ca7 (GAGAGAAGAA GAAAAAAG AGAGGCCTAT TTGGGG) (SEQ ID NO: 5) in case of the HA gene of A/California/07/09. In case of A/Singapore/1/1957 the oligo pairs o.HA-Si5 (GAAGGCCGTCAAGGCCGCATGG) (SEQ ID NO: 6) plus o.HA-Si8 (TCTCTTTTTT CTTCTTCTCT CTCTTGATTC AATCTGGGGA ACATT) (SEQ ID NO: 7) and o.HA-Si6 (GCAGTGAAAG GAAGGCCCAT GAGG) (SEQ ID NO: 8) plus o.HA-Si7 (GAGAGAAGAA GAAAAAAGAG AGGATTGTTT GGGGCAATAG C) (SEQ ID NO: 9) are used. After purification (Sigma GENELUTE™, Sigma, Inc.) the two PCR fragments are used to amplify the complete CDS of the modified HA (mHA) with the oligos o.HA-Ca5 and o.HA.Ca6 or o.HA-Si5 and o.HA-Si6. The mHA gene cassette of A/California/07/09 is introduced into the plasmid pHA-mH5-H1-Ca by substituting the wild-type HA gene cassette using the restriction sites NheI and NotI resulting in pHA-mH5-mHA-Ca and pHA-mH5-mHA-Si, respectively. All plasmids are verified by sequencing. Alternatively, the full sequence of the modified HA-gene cassettes are synthesized and then cloned into the appropriate transfer plasmids.

Construction and characterization of recombinant MVA viruses. MVA-mHA-Ca and MVA-mHA-Si. Twenty micrograms of pHA-mH5-mHA-Ca or pHA-mH5-mHA-Si plasmid DNA are transfected into MVA-infected chicken cells by calcium phosphate precipitation and further processed as described previously (14). The purified recombinant virus isolates are expanded for large scale preparations in chicken cells and characterized by PCR or by Southern blotting according to standard procedures.

Expression of the H1, H2 and mHA proteins by recombinant MVA is detected by Western blotting. DF-1 cells are infected at a multiplicity of infection of 0.1 for 48 h. Infected cells are harvested by scraping or by adding trypsin. Sonicated cell lysates are loaded onto 12% polyacrylamide gels (BioRad, Inc) and afterwards blotted on nitrocellulose membrane (Invitrogen, Inc). To detect the H1 protein, a sheep antiserum against the A/California/7/2009 hemagglutinin (NIBSC 09/152) is used. Donkey-anti-sheep alkaline phosphatase-conjugated IgG (Sigma Inc.) is used as a secondary antibody.

### Example 3

### Construction and Use of a MVA Virus Expressing the Swine Influenza HA and NA Genes (H1N1)

Since both HA and NA contribute to protection, a MVA live vaccine (termed ‘MVA-H1-N1-Ca’) containing both antigens is useful to achieve greater immunogenicity. To generate this virus, a double gene cassette is constructed that contains the HA gene cassette (consisting of a first vaccinia virus promoter and the HA coding sequence) and the NA gene cassette (consisting of a second vaccinia virus promoter and the NA coding sequence). Vaccinia promoters are preferably strong early/late promoters such as the modified H5 promoter (22) or a synthetic early/late promoter (25) or the P7.5 early late promoter (26).

A plasmid containing the double gene cassette is used to construct the virus MVA-H1-N1-Ca by in-vivo recombination techniques including selection and/or rescue techniques (27, 5, 14). Western blotting is used to verify expression of the HA and NA proteins in infected cells as in the previous example. The resulting virus MVA-H1-N1-Ca induces strong neutralizing and neuraminidase inhibiting (NI) antibodies. In addition, strong CD8 T cell responses are induced against the NA and HA antigens. The structure of the recombinant virus is shown in FIG. 10 and FIG. 11.

Materials and Methods: Cloning of the plasmids containing the double gene cassette. First, a plasmid was cloned, which contains the NA gene of the A/California/04/09 strain downstream of the synthetic early/late promoter selP. For this purpose, the N1 gene (excised from pHA-mH5-NA-Ca; see example 1) was inserted into the plasmid pDM-D4R (Ricci, University of Vienna) using the restriction enzymes Eco105I and NotI. The resulting plasmid was termed pDM-N1ca. The plasmid directs the gene cassette into the D4/D5 intergenic region of MVA. The HA gene cassette containing the hemagglutinin sequences of A/California/04/2009 (see Example 1) was chemically synthesized and cloned in pDM-N1ca using the restriction sites XhoI and NheI. The resulting plasmid containing the wild-type HA and NA sequence of A/California/04/09 was termed pDM-H1-N1ca.

Results

Construction and characterization of the double recombinant MVA virus MVA-H1-N1-Ca: The NA gene of A/California/07/09 (H1N1) was placed downstream the synthetic early/late promoter selP while the HA gene was under control of the strong early/late promoter mH5. The resulting plasmid containing the genes in tandem order was used to construct the virus MVA-H1-N1ca by in-vivo recombination techniques as described in Example 1. The titer of the final virus preparation was 3.2×10⁹ pfu/ml. The virus construct was characterized by PCR for absence of wild-type virus and for presence of the HA and NA gene inserts.

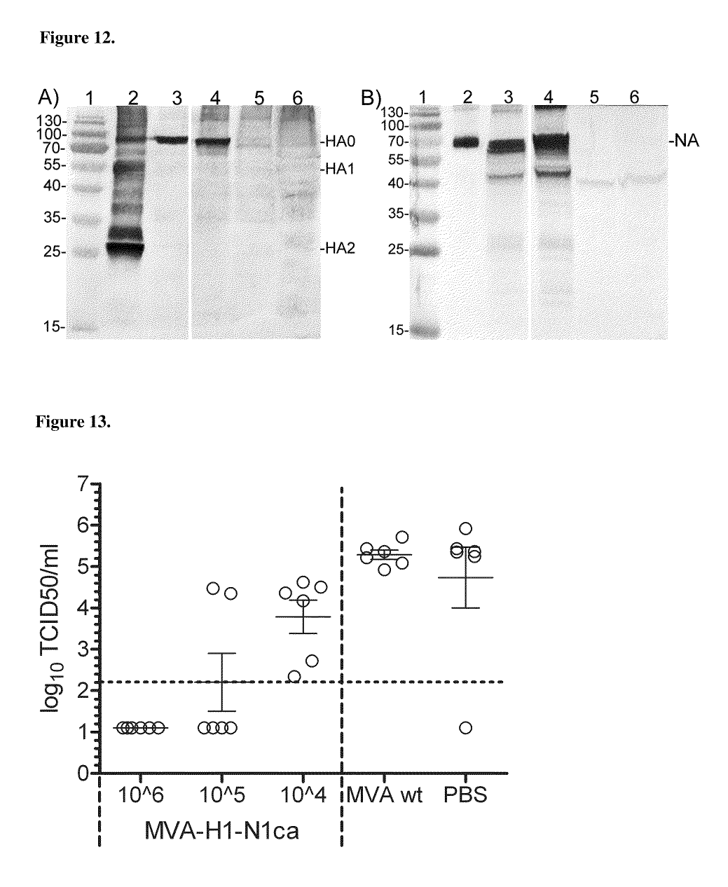

Subsequently, the correct expression of the influenza HA and NA genes was analyzed. Total cell lysates were analyzed by SDS-PAGE and Western blotting using anti-hemagglutinin A/California/7/09 (H1N1) polyclonal serum or anti-neuraminidase antibodies. As controls, cell lysates infected with MVA-H1-Ca or MVA-N1ca (see Example 1) were used. Both recombinant viruses express the wild-type HA and NA protein of A/California/07/2009 (H1N1), respectively. Furthermore, an inactivated whole virus vaccine of H1N1 (Kistner et al., PLoS One 5:e9349, 2010), and an uninfected cell lysates were used. The Western Blot probed with the HA anti-serum demonstrated that the double recombinant MVA-H1-N1ca expresses the HA protein (FIG. 12A). The large bands at 80 kDa represent the uncleaved HA0, which is the exclusive form of HA after infection of avian DF-1 cells with MVAs coding the wild-type HA protein. The lower bands around 55 and 26 kDa represent the HA1 and HA2 subunits and are seen in the inactivated H1N1 control.

To reveal the expression of the neuraminidase, the Western Blot was probed with an antibody raised against a peptide present in the neuraminidase of different subtypes including the N1. The double construct induced a band around 75 kDa similar to the MVA-N1-Ca (Example 1) and the inactivated H1N1 vaccine (FIG. 12B). The specific band is absent in the wild-type MVA control, and uninfected cell lysates.

Protection studies in immune competent mice: Protection studies were carried out in BALB/c mice similar as described in Example 1. Mice were immunized once with three different doses (10⁴, 10⁵, 10⁶ pfu per animal) of MVA-H1-N1ca. At day 42 they were challenged intranasally with 10⁵ TCID₅₀ of wild-type virus per animal. Three days later lungs were removed and lung titers were determined by TCID₅₀ titration. Protection results are compiled in Table 6 and displayed in FIG. 13. Mice vaccinated with the controls (wild-type MVA (MVA wt) or PBS) were not protected showing average log 10 TCID₅₀ titers of 5.3 or 4.7. Mice vaccinated with 10⁶ pfu of MVA-H1-N1-Ca were fully protected. Partial protection was achieved with 10⁴ or 10⁵ pfu MVA-H1-N1-Ca. The experiment shows that a single dose of virus as low as 10⁴ or 10⁵ pfu per mouse results in partial protection against lung viremia and a single dose of 10⁶ results in full protection. Compared to the previously obtained results (see Example 1), the double insert virus shows a more robust protection.

TABLE 6
Protection of mice from lung viremia after
single dose vaccinations with MVA-H1-N1ca.

| | | | | Average | |
|---|---|---|---|---|---|
| | | Vaccine | | lung titer | |
| | | dose | HI | (log₁₀ | Protection |
| Gr. | Vaccine | [pfu/mouse] | titer⁽¹⁾ | TCID₅₀) | n/nt (%) |
| 1 | MVA-H1-N1ca | 10⁴ | 20 | <dl | 0/6 (0) |
| 2 | MVA-H1-N1ca | 10⁵ | 20 | 2.2 | 4/6 (66.7) |
| 3 | MVA-H1-N1ca | 10⁶ | 160 | 3.8 | 6/6 (100) |
| 4 | MVA-wt⁽²⁾ | 10⁷ | <dl | 5.3 | 0/6 (0) |
| 5 | PBS | 10⁶ | <dl | 4.7 | 0/6 (0) |

⁽¹⁾hemagglutinin-inhibition titer determined with chicken erythrocytes; dl, detection limit of the assay was of log10 2.21;
⁽²⁾wild-type MVA (NIH74 LVD clone 6).

### Example 4

### Construction and Use of a MVA Virus Expressing the Swine Flu HA, NA and NP Gene

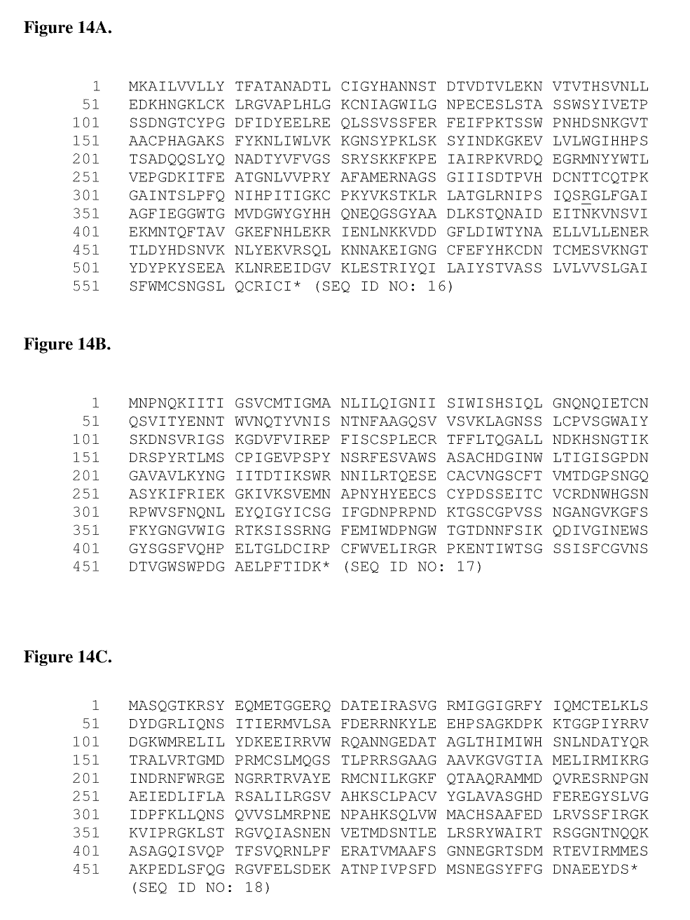

Since HA, NA and NP contribute to protection and cross-protection, an MVA live vaccine (termed ‘MVA-H1-N1-NP’) containing the three antigens provides another technique for generating immunity to influenza virus. To generate this recombinant virus, a plasmid is constructed that contains the NP gene cassette (consisting of a vaccinia virus promoter and the NP coding sequence) which is inserted into the MVA-H1N1-Ca virus. A transfer plasmid is chosen, that directs the gene cassette into the deletion III region of MVA or into the vaccinia HA-locus (2). Alternatively, co-infection of the MVA-H1N1 virus with a MVA-NP virus and screening for the virus with the three inserts can be carried out. Western blotting is used to verify expression of the HA, NA and NP proteins in infected cells. The virus MVA-H1N1-NP induces strong neutralizing and neuraminidase inhibiting (NI) antibodies. In addition, strong CD8 T cell responses are induced against all three influenza genes. This virus protects animals from lethal challenge with swine flu influenza viruses. The structure of this virus is shown in FIG. 11 and the sequences used in FIGS. 14A to 14C.

Cloning of the NP gene. The nucleoprotein sequence (accession number FJ966083) was chemically synthesized (Geneart, Inc., Germany) and the recombinant gene is operably liked to the mH5 vaccinia virus promoter and terminated with a vaccinia virus specific stop signal downstream of the coding region that is absent internally. The expression cassette was cloned into the MVA transfer plasmid pd3-lacZ-gpt using the restriction sites SpeI and NotI, resulting in the vector pD3-NPca. The insertion plasmid directs the gene cassettes into the MVA dIII-locus (Meyer et al., J. Gen. Virol. 72:1031-1038, 1991; Antoine et al., Virology 244:365-396, 1998). The vector further comprises a marker cassette consisting of a LacZ gene and a gpt gene.

Construction and characterization of recombinant MVA virus MVA-H1-N1-NPca. Twenty micrograms of pD3-NPca plasmid DNA were transfected into MVA-H1-N1ca infected chicken cells by calcium phosphate precipitation and plaques were isolated and purified according to standard procedures. The plaque-purified recombinant virus isolates were expanded for large scale preparations in CEC and purified by centrifugation through a sucrose cushion.

Western Blot analysis. To detect the expression of NP, confluent DF-1 were infected with 0.1 MOI of MVA-H1-N1-NPca for 48 h. Infected cells were harvested by scraping, mixed with 2× Laemmli buffer (Fermentas, Glen Burnie, Md.), and sonicated. The cell lysates, together with controls, were loaded onto 12% polyacrylamide gels (BioRad, Inc.) and afterwards blotted on nitrocellulose membrane (Invitrogen, Inc.). To detect the NP protein, a 1:100 dilution of a polyclonal mouse influenza serum was used. A 1:2000 diluted goat-anti-mouse alkaline phosphatase-conjugated IgG (Sigma) was used as secondary antibody. Expression of the N1 and the wild-type H1 proteins by the triple recombinant MVAs was detected by Western blotting as described above.

The triple recombinant virus MVA-H1-N1-Npca was constructed as described above by inserting the NP gene into the pre-existing double insert virus MVA-H1-N1ca. The virus construct was characterized by PCR for absence of wild-type virus and for the presence of the HA, NA, and NP gene inserts. Furthermore, after plaque screening, expression of NP was shown by western blotting with a human anti-NP specific antibody in the single plaque isolates. One of the NP-positive clones was amplified and called MVA-H1-N1-Npca.

The correct expression of the influenza HA, NA and NP genes by the purified triple recombinant MVA is analyzed by Western blotting. Total cell lysates are analyzed by SDS-PAGE and Western blotting using anti-hemagglutinin A/California/7/09 (H1N1) polyclonal serum, or anti-neuraminidase antibody, or a polyclonal human serum. As a control, cell lysates infected with the dedicated single and double recombinants (see Examples 1-3) are used. Furthermore, the inactivated whole virus vaccine (Kistner et al., PLoS One 5:e9349, 2010), and uninfected cell lysates are used.

Protection studies with the triple virus are performed showing that immune responses against HA (neutralizing antibodies), against NA (NA-inhibiting antibodies) and against NP (CD8 T cell responses) are induced.

### Example 5

### Construction of MVA-H2-Singapore

The influenza subtype causing the next pandemic is unknown. Virus subtypes that caused pandemics decades ago may re-appear when immunity against the specific subtype in the human population has waned. A candidate of this kind may be the H2 subtype with the A/Singapore/1/57(H2N2) strain as a prototype. Therefore, a pre-pandemic MVA vaccine based on the Singapore H2 strain was constructed. First, the H2 gene was inserted into a transfer plasmid that directs the H2 into the vaccinia HA-locus. Then, the recombinant MVA virus was constructed and plaque-purified three times. Western blot confirmed expression of the H2 as a single band in the 60 kDa size range (see Materials and Methods). Protective efficacy is shown is mouse, ferret and guinea pig animal models.

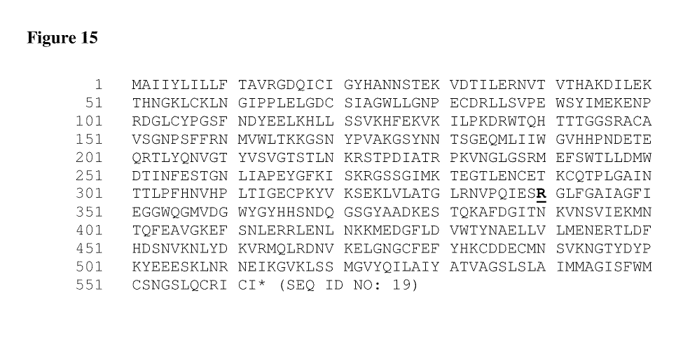

Construction and characterization of recombinant vaccinia virus. The hemagglutinin (Genbank accession #CY034044) sequence of A/Singapore/1/1957 (FIG. 15) was synthesized by Geneart (Regensburg, Germany). The synthetic gene includes the strong early/late vaccinia promoter mH5 upstream of the coding region and a vaccinia virus specific stop signal downstream of the coding region. For the MVA insertion plasmids, the gene cassette of HA was cloned into pHA-vA (14), resulting in pHA-mH5-HA-Si. The insertion plasmid directs the gene cassettes into the MVA HA-locus.

Twenty micrograms of pHA-mH5-HA-Si plasmid DNA was transfected into MVA infected primary chicken cells by calcium phosphate precipitation and further processed as described previously (14). The purified recombinant virus isolate was expanded for large scale preparations in primary chicken cells (CEC) and purified by centrifugation through sucrose cushion.

Western Blot analysis. To reveal the expression of the HA protein, confluent DF-1 were infected with 0.1 MOI of MVA-H2-Si for 48 hours. Infected cells were harvested by scraping, mixed with 2x Laemmli buffer (Fermentas), and sonicated. The cell lysates, together with controls, were loaded onto 12% polyacrylamide gels (BioRad, Inc.) and afterwards blotted on nitrocellulose membrane (Invitrogen, Inc.). To detect the H2 protein, a 1:1000 dilution of a sheep antiserum against the A/California/7/2009 HA (NIBSC 09/152) was used. A 1:2000 diluted donkey-anti-sheep alkaline phosphates-conjugated IgG (Sigma Inc.) was used as a secondary antibody. A whole virus vaccine H1N1 A/California/7/2009 served as positive control.

The virus construct was characterized by PCR for absence of wild-type virus and for presence of the HA gene. The correct expression of the influenza H2 protein by the recombinant MVA was analyzed by Western blotting as described above. Total cell lysates were analyzed by SDS-PAGE and Western blotting using anti-hemagglutinin A/California/7/09 (H1N1) polyclonal serum. Cell lysates infected with the wild type MVA, uninfected cell lysates, and an inactivated whole virus vaccine of H1N1 were used as controls. The Western Blot probed with the HA serum demonstrated the correct expression of the uncleaved HA0 represented by large bands at 80 kDa. Due to lack of the polybasic cleavage site, the HA0 precursor protein was not cleaved by intracellular proteases produced by the avian DF-1 cells. The large 80 kDa band represents the uncleaved HA0. The specific HA bands were absent in the control lysates.

These results show that the virus is a suitable vaccine candidate against the H2 influenza viruses. Similar to the previous examples, double or triple constructs expressing, in addition, the NA and NP genes are constructed, that expand the protection against potential pandemic H2 outbreaks and, due to the common NP antigen, against influenza A viruses in general.

Numerous modifications and variations in the invention as set forth in the above illustrative examples are expected to occur to those skilled in the art. Consequently only such limitations as appear in the appended claims should be placed on the invention.

### REFERENCES

1. Alexandrova, G. I., F. I. Polezhaev, G. N. Budilovsky, L. M. Garmashova, N. A. Topuria, A. Y. Egorov, Y. R. Romejko-Gurko, T. A. Koval, K. V. Lisovskaya, A. I. Klimov, and et al. 1984. Recombinant cold-adapted attenuated influenza A vaccines for use in children: reactogenicity and antigenic activity of cold-adapted recombinants and analysis of isolates from the vaccinees. Infect. Immun. 44:734-9.
2. Antoine, G., F. Scheiflinger, F. Dorner, and F. G. Falkner. 1998. The complete genomic sequence of the modified vaccinia Ankara strain: comparison with other orthopoxviruses. Virology 244:365-396.
3. Gubareva, L. V., J. M. Wood, W. J. Meyer, J. M. Katz, J. S. Robertson, D. Major, and R. G. Webster. 1994. Codominant mixtures of viruses in reference strains of influenza virus due to host cell variation. Virology 199:89-97.
4. Himly, M., D. N. Foster, I. Bottoli, J. S. Iacovoni, and P. K. Vogt. 1998. The DF-1 chicken fibroblast cell line: transformation induced by diverse oncogenes and cell death resulting from infection by avian leukosis viruses. Virology 248:295-304.
5. Holzer, G. W., W. Gritschenberger, J. A. Mayrhofer, V. Wieser, F. Dorner, and F. G. Falkner. 1998. Dominant host range selection of vaccinia recombinants by rescue of an essential gene. Virology 249:160-166.
6. Karron, R. A., K. Talaat, C. Luke, K. Callahan, B. Thumar, S. Dilorenzo, J. McAuliffe, E. Schappell, A. Suguitan, K. Mills, G. Chen, E. Lamirande, K. Coelingh, H. Jin, B. R. Murphy, G. Kemble, and K. Subbarao. 2009. Evaluation of two live attenuated cold-adapted H5N1 influenza virus vaccines in healthy adults. Vaccine 27:4953-4960.
7. Kilbourne, E. D., W. G. Laver, J. L. Schulman, and R. G. Webster. 1968. Antiviral activity of antiserum specific for an influenza virus neuraminidase. J. Virol. 2:281-288.
8. Kistner, O., B. A. Crowe, W. Wodal, A. Kerschbaum, H. Savidis-Dacho, N. Sabarth, F. G. Falkner, I. Mayerhofer, W. Mundt, M. Reiter, L. Grillberger, C. Tauer, M. Graninger, A. Sachslehner, M. Schwendinger, P. Brühl, T. R. Kreil, H. J. Ehrlich, and P. N. Barrett. submitted. A Whole Virus Pandemic Influenza H1N1 Vaccine is Highly Immunogenic and Protective in Active Immunization and Passive Protection Mouse Models. PLOs One.
9. Kistner, O., M. K. Howard, M. Spruth, W. Wodal, P. Bruhl, M. Gerencer, B. A. Crowe, H. Savidis-Dacho, I. Livey, M. Reiter, I. Mayerhofer, C. Tauer, L. Grillberger, W. Mundt, F. G. Falkner, and P. N. Barrett. 2007. Cell culture (Vero) derived whole virus (H5N1) vaccine based on wild-type virus strain induces cross-protective immune responses. Vaccine 25:6028-6036.
10. Maassab, H. F., and M. L. Bryant. 1999. The development of live attenuated cold-adapted influenza virus vaccine for humans. Rev Med Virol 9:237-44.
11. Maines, T. R., A. Jayaraman, J. A. Belser, D. A. Wadford, C. Pappas, H. Zeng, K. M. Gustin, M. B. Pearce, K. Viswanathan, Z. H. Shriver, R. Raman, N. J. Cox, R. Sasisekharan, J. M. Katz, and T. M. Tumpey. 2009. Transmission and pathogenesis of swine-origin 2009 A(H1N1) influenza viruses in ferrets and mice. Science 325:484-7.
12. Palese, P., T. Muster, H. Zheng, R. O'Neill, and A. Garcia-Sastre. 1999. Learning from our foes: a novel vaccine concept for influenza virus. Arch. Virol. Suppl. 15:131-138.
13. Ramirez, J. C., M. M. Gherardi, D. Rodriguez, and M. Esteban. 2000. Attenuated modified vaccinia virus Ankara can be used as an immunizing agent under conditions of preexisting immunity to the vector. J. Virol. 74:7651-7655.
14. Scheiflinger, F., F. Dorner, and F. G. Falkner. 1998. Transient marker stabilisation: a general procedure to construct marker-free recombinant vaccinia virus. Arch. Virol. 143:467-74.
15. Scherle, P. A., G. Palladino, and W. Gerhard. 1992. Mice can recover from pulmonary influenza virus infection in the absence of class I-restricted cytotoxic T cells. J. Immunol. 148:212-217.
16. Seo, S. H., M. Peiris, and R. G. Webster. 2002. Protective cross-reactive cellular immunity to lethal A/Goose/Guangdong/1/96-like H5N1 influenza virus is correlated with the proportion of pulmonary CD8(+) T cells expressing gamma interferon. J. Virol. 76:4886-4890.
17. Suguitan, A. L., Jr., M. P. Marino, P. D. Desai, L. M. Chen, Y. Matsuoka, R. O. Donis, H. Jin, D. E. Swayne, G. Kemble, and K. Subbarao. 2009. The influence of the multi-basic cleavage site of the H5 hemagglutinin on the attenuation, immunogenicity and efficacy of a live attenuated influenza A H5N1 cold-adapted vaccine virus. Virology 395:280-288.
18. Talon, J., M. Salvatore, R. E. O'Neill, Y. Nakaya, H. Zheng, T. Muster, A. Garcia-Sastre, and P. Palese. 2000. Influenza A and B viruses expressing altered NS1 proteins: A vaccine approach. Proc Natl Acad Sci USA 97:4309-14.
19. Williams, S. P., and J. S. Robertson. 1993. Analysis of the restriction to the growth of nonegg-adapted human influenza virus in eggs. Virology 196:660-665.
20. Wood, J. M., and J. S. Robertson. 2004. From lethal virus to life-saving vaccine: developing inactivated vaccines for pandemic influenza. Nat. Rev. Microbiol. 2:842-847.
21. Wright, P. F., G. Neumann, and Y. Kawaoka (ed.). 2007. Orthomyxoviruses. Fields Virology, 5th Edition, vol. 2. Lippincott Williams & Wilkins.
22. Wyatt, L. S., S. T. Shors, B. R. Murphy, and B. Moss. 1996. Development of a replication-deficient recombinant vaccinia virus vaccine effective against parainfluenza virus 3 infection in an animal model. Vaccine 14:1451-1458.
23. Yap, K. L., and G. L. Ada. 1978. The recovery of mice from influenza A virus infection: adoptive transfer of immunity with influenza virus-specific cytotoxic T lymphocytes recognizing a common virion antigen. Scand. J. Immunol. 8:413-420.
24. Antoine, G., F. Scheiflinger, F. Dorner, and F. G. Falkner. 1998. The complete genomic sequence of the modified vaccinia Ankara strain: comparison with other orthopoxviruses. Virology 244:365-396.
25. Chakrabarti, S., J. R. Sisler, and B. Moss. 1997. Compact, synthetic, vaccinia virus early/late promoter for protein expression. Biotechniques 23:1094-7.
26. Coupar, B. E., M. E. Andrew, G. W. Both, and D. B. Boyle. 1986. Temporal regulation of influenza hemagglutinin expression in vaccinia virus recombinants and effects on the immune response. Eur J Immunol 16:1479-87.
27. Falkner, F. G., and B. Moss. 1988. Escherichia coli gpt gene provides dominant selection for vaccinia virus open reading frame expression vectors. J Virol 62:1849-54.
28. Holzer, G. W., and F. G. Falkner. 1997. Construction of a vaccinia virus deficient in the essential DNA repair enzyme uracil DNA glycosylase by a complementing cell line. J. Virol. 71:4997-5002
29. Mayrhofer, J., S. Coulibaly, A. Hessel, G. W. Holzer, M. Schwendinger, P. Bruhl, M. Gerencer, B. A. Crowe, S. Shuo, W. Hong, Y. J. Tan, B. Dietrich, N. Sabarth, H. Savidis-Dacho, O. Kistner, P. N. Barrett, and F. G. Falkner. 2009. Nonreplicating vaccinia virus vectors expressing the H5 influenza virus hemagglutinin produced in modified Vero cells induce robust protection. J Virol 83:5192-203.
30. Stech, O., J. Veits, S. Weber, D. Deckers, D. Schroer, T. W. Vahlenkamp, A. Breithaupt, J. Teifke, T. C. Mettenleiter, and J. Stech. 2009. Acquisition of a polybasic hemagglutinin cleavage site by a low-pathogenic avian influenza virus is not sufficient for immediate transformation into a highly pathogenic strain. J Virol 83:5864-8.
31. Sutter, G., and B. Moss. 1992. Nonreplicating vaccinia vector efficiently expresses recombinant genes. Proc Natl Acad Sci USA 89:10847-51.
32. Yen, H. L., and R. G. Webster. 2009. Pandemic influenza as a current threat. Curr Top Microbiol Immunol 333:3-24.

1. An antigenic composition comprising a vaccinia virus vector comprising a polynucleotide encoding a hemagglutinin protein (HA) from influenza A and a polynucleotide encoding a neuraminidase protein (NA) from an influenza A virus, wherein the HA is of subtype selected from the group consisting of H1, H2, H3, H4, H5, H6, H7, H8, H9, H10, H11, H12, H13, H14, H15, and H16, and wherein the NA is of a subtype selected from the group consisting of N1, N2, N3, N4, N5, N6, N7, N8 and N9.

2. The antigenic composition of claim 1, wherein the polynucleotide encoding the HA and the polynucleotide encoding the NA are derived from the same virus strain.

3. The antigenic composition of claim 1, wherein the polynucleotide encoding the HA and the polynucleotide encoding the NA are derived from different virus strains.

4. The antigenic composition of claim 1, wherein the HA is derived from subtype H1 and the NA derived from subtype N1.

5. The antigenic composition of claim 4, wherein the HA and NA are derived from influenza A strain virus A/California/07/2009.

6. The antigenic composition of claim 5, wherein the HA protein encoded by the polynucleotide is set out in FIG. 8 (SEQ ID NO: 14) or FIG. 14A (SEQ ID NO: 16).

7. The antigenic composition of claim 5, wherein the NA protein encoded by the polynucleotide is set out in FIG. 14B (SEQ ID NO: 17).

8. The antigenic composition of claim 1, wherein the HA is of subtype H2.

9. The antigenic composition of claim 1, wherein the vector optionally comprises a polynucleotide encoding an influenza A nucleoprotein (NP) protein.

10. The antigenic composition of claim 9, wherein the HA, NA and NP are derived from the same strain.

11. The antigenic composition of claim 1, wherein the vaccinia virus is selected from the group consisting of modified vaccinia Ankara (MVA), vaccinia virus Lister (VVL) and defective vaccinia Lister.

12. The antigenic composition of claim 1, said antigenic composition characterized by the ability to propagate in vertebrate cell culture.

13. The antigenic composition of claim 12, wherein the vertebrate cell is selected from the group consisting of MRC-5, Vero, CV-1, MDCK, MDBK, HEK, H9, CEM, PerC6, BHK-21, BSC and LLC-MK2, DF-1, QT-35, or primary chicken cells.

14. The antigenic composition of claim 13, wherein the vertebrate cell is a Vero cell.

15. The antigenic composition of claim 1, further comprising a pharmaceutically acceptable carrier.

16. The antigenic composition of claim 1, wherein the HA comprises a polybasic cleavage site.

17. The antigenic composition of claim 16, wherein the polybasic cleavage site has the amino acid sequence RERRRKKR (SEQ ID NO: 1).

18. A recombinant vaccinia virus comprising a polynucleotide encoding a hemagglutinin protein (HA) from influenza A and a polynucleotide encoding a neuraminidase protein (NA) from an influenza A virus, wherein the HA is of subtype selected from the group consisting of H1, H2, H3, H4, H5, H6, H7, H8, H9, H10, H11, H12, H13, H14, H15, and H16, and wherein the NA is of a subtype selected from the group consisting of N1, N2, N3, N4, N5, N6, N7, N8 and N9.

19-27. (canceled)

28. The recombinant vaccinia virus of claim 18, wherein the vaccinia virus is selected from the group consisting of modified vaccinia Ankara (MVA), vaccinia virus Lister (VVL) and defective vaccinia Lister.

29. The recombinant vaccinia virus of claim 18, said recombinant vaccinia virus characterized by the ability to propagate in vertebrate cell culture.

30-34. (canceled)

35. A vaccine comprising: a vaccinia virus vector comprising a polynucleotide encoding a hemagglutinin protein (HA) from influenza A and a polynucleotide encoding a neuraminidase protein (NA) from an influenza A virus, wherein the HA is from a subtype selected from the group consisting of H1, H2, H3, H4, H5, H6, H7, H8, H9, H10, H11, H12, H13, H14, H15, and H16, and wherein the NA is from a subtype selected from the group consisting of N1, N2, N3, N4, N5, N6, N7, N8 and N9, and
wherein the polynucleotide encoding the HA and the polynucleotide encoding the NA are operatively linked to allow packaging of the polynucleotides into a virion.

36. A vaccine comprising, i) a first vaccinia virus vector comprising a polynucleotide encoding a hemagglutinin protein (HA) from influenza A, and ii) a second vaccinia vector comprising a polynucleotide encoding a neuraminidase protein (NA) from an influenza A virus,
wherein the polynucleotide encoding the HA and the polynucleotide encoding the NA are operatively linked to allow packaging of the polynucleotides into a virion.

37-46. (canceled)

47. The vaccine of any one of claim 35 or 36, wherein the polynucleotide encoding the HA, the polynucleotide encoding the NA and the polynucleotide encoding the NP are each operably linked to a promoter.

48-53. (canceled)

54. A method for eliciting an immune response against at least one influenza virus strain in a subject, comprising administering an antigenic composition of claim 1, a recombinant vaccinia virus of claim 18 or a vaccine of claim 35 or claim 36 in an amount effective to elicit the immune response against at least one influenza virus strain.

55. A method for preventing infection of a subject by an influenza virus comprising, administering to the subject an effective amount of an antigenic composition of claim 1, a recombinant vaccinia virus of claim 18 or a vaccine of claim 35 or claim 36 in an amount effective to prevent infection of the subject by the influenza virus.

56-57. (canceled)

58. A method of making a vaccine comprising a vaccinia virus vector and polynucleotide encoding a hemagglutinin protein (HA) from influenza A and a polynucleotide encoding a neuraminidase protein (NA) from an influenza A virus, wherein the HA is of subtype selected from the group consisting of H1, H2, H3, H4, H5, H6, H7, H8, H9, H10, H11, H12, H13, H14, H15, and H16, and wherein the NA is of a subtype selected from the group consisting of N1, N2, N3, N4, N5, N6, N7, N8 and N9, and optionally comprising a polynucleotide encoding an influenza A nucleoprotein (NP) protein, the method comprising transfecting the HA, NA, and optionally NP, polynucleotides into the virus in vertebrate cells under conditions suitable for growth of the virus.

59-69. (canceled)
